(19) United States
(12) Patent Application Publication
(10) Pub. No.: US 2023/0277106 A1
(43) Pub. Date: Sep. 7, 2023

(54) HOT FLASH MULTI-SENSOR CIRCUIT SYSTEM

(71) Applicant: SRI International, Menlo Park, CA (US)

(72) Inventors: Massimiliano de Zambotti, Sunnyvale, CA (US); Fiona Baker, San Jose, CA (US); David Smith, Ukiah, CA (US); Andreas Tsiartas, San Jose, CA (US)

(73) Assignee: SRI International, Menlo Park, CA (US)

(21) Appl. No.: 18/019,893

(22) PCT Filed: Aug. 6, 2021
(86) PCT No.: PCT/US2021/044980

§ 371 Date: Feb. 6, 2023

Related U.S. Application Data
(60) Provisional application No. 63/062,692, filed on Aug. 7, 2020.

(51) Int. Cl. A61B 5/16; A61B 5/00; G16H 50/20

(52) U.S. Cl. CPC A61B 5/165; A61B 5/7264; G16H 50/20

(57) ABSTRACT

Embodiments in accordance with the present disclosure are directed to systems, devices, and methods involving hot flash (HF) multi-sensor circuits. An example system includes a plurality of sensor circuits and processor circuitry. The sensor circuits obtain a plurality of sensor signals associated with the user. The processor circuitry extracts features from the plurality of sensor signals obtained by the plurality of sensor circuits, aligns the extracted features to a common time point, identifies a HF event for the user using a predictive data model indicative of probability of the HF event occurring for the user at a date and time and based on the aligned extracted features, and communicates a message indicative of the HF event to the user.

### OVERVIEW

Hot flashes (HFs), also called vasomotor symptoms or hot flush, are a sensation of heat, sweating, flashing, anxiety, and chills that generally last between three to ten minutes. HFs are common in women approaching menopause and post-menopause. For example, it is estimated that up to eighty percent of women reaching menopause are plagued by HFs, which can persist for several years post-menopause. Some women have HFs hourly or daily, and others report one or two per week. HFs are not limited to women approaching menopause or post-menopause. For example, women who have a hysterectomy and ovariectomy or women who are undergoing certain treatments for breast cancer may also experience frequent and severe HFs as one of their symptoms. Additionally, men may experience HFs when undergoing certain treatments, such as cancer related treatment.

Menopausal HFs occur in association with a shift in reproductive hormone levels, with an increase in follicle stimulating hormone and a decrease in estradiol in the approach to menopause. This withdrawal of estrogen is hypothesized to impact the stability of the central thermoregulatory center in the brain, leading to the manifestation of HFs. Alteration in automatic nervous system controls may also be implicated in the manifestation of the vasomotor symptoms. HFs negatively impact daytime functioning, work productivity, mood, and sleep, and are linked with increased risk for cardiovascular disease in later life. Since HFs can persist for several years past menopause, HFs potentially have a long-term negative influence on quality of life.

An underlying distressing factor of HFs is that the sufferer has little control over HFs as HFs can seem to occur at random inconvenient times, day and night, interfering with work, home, and sleep. HFs can be eradicated with hormone therapy, but hormone therapy is not appropriate for everyone, whether due to risk profiles or personal preferences. Other non-hormonal prescription medications that can be effective include selective serotonin re-uptake inhibitors and gabapentin, but these treatments also have side-effects and are not for everyone. There are also non-pharmacological options which may focus on the negative consequences of HFs (e.g., discomfort due to sweating, irritation/anxiety due to the embarrassment of experiencing hot flashes in public or during work) as a single time point intervention after a HF manifests.

### SUMMARY OF THE INVENTION

The present invention is directed to overcoming the above-mentioned challenges and others related to identifying and/or managing HFs.

Various embodiments are directed to a system including a plurality of sensor circuits and processor circuitry, such as a HF management system and/or a menopause management system. The plurality of sensor circuits are configured to obtain a plurality of sensor signals associated with the user. The sensor signals can be indicative of physiological measurements of the user. The processor circuitry is in communication with the plurality of sensor circuits and configured to extract features from the plurality of sensor signals obtained by the plurality of sensor circuits, align the extracted features to a common time point, identify a HF event for the user using a predictive data model indicative of a probability of the HF event occurring for the user at a date and time and based on the aligned extracted features, and communicate a message indicative of the HF event to the user.

In some embodiments, the plurality of sensor circuits include two or more sensor circuits selected from a photoplethysmogram (PPG) sensor, a skin conductance (SC) sensor, a temperature (T) sensor, and a motion (M) sensor. In some embodiments, the plurality of sensor circuits include each of a PPG sensor, a SC sensor, a T sensor, and a M sensor.

In some embodiments, the processor circuitry is configured to characterize a level or a presence of the HF event based on the extracted features.

In some embodiments, the message includes at least one of the identification of the HF event and an intervention action for the HF event, and the processor circuitry is configured to identify the HF event in real-time.

In some embodiments, the processor circuitry is configured to identify a psychophysiological state of the user based on the extracted features, and identify, using the predictive data model, a pattern of physiological measurements indicative of the probability of the HF event occurring based on the aligned extracted features and the psychophysiological state of the user. The psychophysiological state can include a sleep state or an awake state, and the processor circuitry can be configured to calculate an amount of awake time associated with (e.g., caused by) the HF event.

In some embodiments, the processor circuitry is configured to align the extracted features to the common time point based on a plurality of different time windows associated with the plurality of sensor circuits, and weigh each of the extracted features based on an impact of the extracted features on the probability of the HF event occurring.

In some embodiments, the predictive data model includes a plurality of sub-models, and each of the plurality of sub-models are associated with a respective sensor circuit of the plurality and each used to provide an output score indicative of the probability of the HF event occurring for the user based on the extracted features from the respective sensor signal obtained by the respective sensor circuit. In such embodiments, the processor circuitry can be configured to combine the output scores from the plurality of sub-models to identify the HF event.

In some embodiments, the processor circuitry is configured to combine the extracted features from the plurality of sensor signals into a vector and input the vector to the predictive data model to produce an output score indicative of the probability.

In some embodiments, the processor circuitry is configured to generate and/or use a decision tree structure to combine the extracted features and to produce the output score based on the combined extracted features. The processor circuitry can further identify whether the HF event is occurring or not at a plurality of time points, detect consecutive identified HF events, and convert the consecutive identified HF events into a HF region.

In some embodiments, the processor circuitry is configured to receive feedback data in response to the communicated message, the feedback data being indicative of at least one of a user confirmation of the HF event, a user denial of the HF event, and a severity of the HF event.

A number of embodiments are directed to non-transitory computer-readable storage medium comprising instructions that when executed cause processor circuitry to extract features from a plurality of sensor signals associated with a user, identify a HF event for the user using a predictive data model indicative of a probability of the HF event occurring for the user at a date and time based on the extracted features, and revise the predictive data model based on feedback data indicative of an impact of the HF event on the user. As previously described, the plurality of sensor signals are obtained by a plurality of sensor circuits.

In some embodiments, the non-transitory computer-readable storage medium further includes instructions executable to align the extracted features to a common time point based on a plurality of different time windows associated with the plurality of sensor circuits used to obtain the plurality of sensor signals.

In some embodiments, the non-transitory computer-readable storage medium further includes instructions executable to receive the feedback data, the feedback data including at least one of a user confirmation of the HF event, a user denial of the HF event, a severity of the HF event, and an impact of an intervention action on the HF event.

In some embodiments, each feature of the extracted features is associated with a weight indicative of an impact of the respective feature on the probability of the HF event. The instructions to revise the predictive data model can include instructions executable to perform at least one of: adjust a first weight associated with a first feature of the extracted features, adjust the first weight and a second weight associated with the first feature for different psychophysiological states of the user, and adjust an intervention action for additional HF events. For example, the features can be used by a binary tree structure in successive thresholds (e.g., higher or lower threshold indicate different weights), which are learned from the data. The thresholds can be adjusted manually and/or automatically by the predictive data model.

In some embodiments, the non-transitory computer-readable storage medium further includes instructions executable to communicate a message indicative of the HF event to the user, wherein the message indicates least one of an occurrence of the HF event, a prediction of the occurrence of the HF event, and an intervention action for the HF event.

Various-related embodiments are directed to a system that includes a plurality of sensor circuits configured to obtain a plurality of sensor signals associated with a user over a plurality of different time windows, and processor circuitry. The processor circuitry is in communication with the plurality of sensor circuits and configured to extract features from the plurality of sensor signals, align the extracted features to a common time point based on the plurality of different time windows associated with the plurality of sensor circuits, track a psychophysiological state of the user based on the aligned extracted features, track a plurality of HF events for the user using a predictive data model indicative of probability of a HF occurring for the user at a date and time based on the aligned extracted features and the tracked psychophysiological state of the user, and communicate a message indicative of the plurality of HF events to the user.

In some embodiments, the tracked psychophysiological state is associated with a sleep state or an awake state of the user, and the processor circuitry is configured to calculate an amount of awake time associated with at least one of the plurality of HF events based on the tracked psychophysiological state.

In some embodiments, the processor circuitry is configured to revise the predictive data model based on feedback data indicative of an impact of the plurality of HF events on the user, the revision including adjusted weights for the extracted features as associated with the tracked psychophysiological state.

In some embodiments, the predictive data model includes weights for each of the extracted features and for each of the different psychophysiological states, each weight being associated with the probability of the HF occurring at the date and time.

Embodiments in accordance with the present disclosure include all combinations of the recited particular embodiments. Further embodiments and the full scope of applicability of the invention will become apparent from the detailed description provided hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description. All publications, patents, and patent applications cited herein, including citations therein, are hereby incorporated by reference in their entirety for all purposes.

### BRIEF DESCRIPTION OF THE DRAWINGS

Various example embodiments may be more completely understood in consideration of the following detailed description in connection with the accompanying drawings, in which:

FIG. 1 illustrates an example of a multi-sensor circuit system for HF identification, in accordance with various embodiments;

FIG. 2 illustrates an example computing device including non-transitory computer-readable medium storing executable code, in accordance with the present disclosure;

FIG. 3 illustrates another example multi-sensor circuit system for HF identification, in accordance with various embodiments;

FIG. 4 illustrates an example of a multi-sensor signal processing, sensor-wise feature extraction, combination, and final HF region(s) extraction, in accordance with various embodiments;

FIG. 5 illustrates an example of a device forming at least part of a system, and processing and output mechanisms, in accordance with various embodiments;

FIG. 6 illustrates an example graph of sensor signals from a plurality of sensor circuits of a system, in accordance with various embodiments;

FIG. 7 illustrates an example graph of SC sensor features as compared to multi-sensor features, in accordance with various embodiments;

FIG. 8 illustrates an example graph of sensor circuit contributions, in accordance with various embodiments;

FIG. 9 illustrates an example graph of system performance for HF onsets during sleep and awake states, in accordance with various embodiments;

FIG. 10 illustrates an example graph of HF classification performance as a function of feature set and corrupted signals, in accordance with various embodiments;

FIGS. 11A-11B illustrate example graphs of sensor circuit contributions during awake and sleep states, in accordance with various embodiments;

FIG. 12 illustrates an example graph of a commercial sensor calibration and conversion, in accordance with various embodiments;

FIGS. 13A-13B illustrate example graphs of a multi-sensor circuit approach versus a gold standard sternum SC approach, in accordance with various embodiments;

FIG. 14 illustrates an example graph of a commercial galvanic skin response (GSR) sensor (applied on the wrist) compared to a gold standard sternum SC approach, in accordance with various embodiments;

FIGS. 15A-15C illustrate example graphs showing experimental results, in accordance with various embodiments; and

FIG. 16 illustrates an example graph of performance of the predictive data model (automatic HF and sleep/wake classification, and automatic calculation of the impact of the HF on sleep), in accordance with various embodiments.

While various embodiments discussed herein are amenable to modifications and alternative forms, aspects thereof have been shown by way of example in the drawings and will be described in detail. It should be understood, however, that the intention is not to limit the invention to the particular embodiments described. On the contrary, the intention is to cover all modifications, equivalents, and alternatives falling within the scope of the disclosure including aspects defined in the claims. In addition, the term “example” as used throughout this application is only by way of illustration, and not limitation.

### DETAILED DESCRIPTION

Aspects of the present disclosure are believed to be applicable to a variety of systems and methods involving a HF identification system that includes multiple sensor circuits. In specific embodiments, the system can include at least two sensor circuits which are used to capture different types of physiological measurements, and the sensor circuits are used in combination with a predictive data model to allow for recovery from erroneous sensor measures. While the present invention is not necessarily limited to such applications, various aspects of the invention may be appreciated through a discussion of various examples using this context.

Accordingly, in the following description various specific details are set forth to describe specific examples presented herein. It should be apparent to one skilled in the art, however, that one or more other examples and/or variations of these examples may be practiced without all the specific details given below. In other instances, well known features have not been described in detail so as not to obscure the description of the examples herein. For ease of illustration, the same reference numerals may be used in different diagrams to refer to the same elements or additional instances of the same element.

HFs are a hallmark symptom of menopause and appear as estrogen levels decline. As women progress through the menopause transition and into post-menopause, the majority of women experience HFs, which are characterized as a sensation of intense heat, sweating, and anxiety, that lasts for five to ten minutes and can occur multiple times across the day and night. HF episodes can persist for several years and can be impactful for women going through menopause: HFs decrease quality of life, interfere with mood, decrease productivity, impair social relationships, impact health, and disrupt sleep. HFs and sleep disturbances are the most common menopausal symptoms for which women seek care. Untreated HFs are associated with higher health care costs and loss of work productivity.

Embodiments in accordance with the present disclosure are directed to a system for identifying and/or managing HFs. In some embodiments, the system is a menopausal management system that aids in the management of menopausal symptoms. However, embodiments are not limited to menopausal women and/or women. The system includes multiple sensor circuits worn by or otherwise associated with the user and, based on the sensor signals, the system identifies a HF event. The HF event can be identified by calculating the probability of a HF occurrence at a particular date and time using data from the multiple sensor circuits. The combination of the sensor circuits can provide data that is comparably reliable to a research-grade SC monitoring devices and can provide greater user compliance and less discomfort than the research-grade SC monitoring devices. The multiple sensors circuits can be used for real-time HF tracking over periods of time due to the increased user comfort, light weight and/or size, data transform protocol, ease of use, and reduced costs as compared to research-grade SC monitoring devices. Additionally, different types of sensor circuits can be used to capture a more complete physiological signature of the HF event and the severity.

Various embodiments are directed to a system that includes a plurality of sensor circuits and processor circuitry that passively identifies HF events from a combination of features extracted from sensor signals recorded by the sensor circuits attached to a body of the user (e.g., wearable device). The processor circuitry can execute or have access to a predictive data model, which can be executed online or off-line and can be adapted to different sensor circuit configurations including the use of contact and non-contact sensors, and with sensors placed in different body locations (e.g., wrist, back of the neck, finger). The processor circuitry reads input data (e.g., sensor signals) streaming from various sensors not limited to: a GSR to measure SC, an inertial measurement unit (IMU) to quantify motion, a skin temperature sensor, and a PPG sensor to measure blood flow. The processor circuitry can log the HF events and/or display the events to the user or potentially actuate a therapeutic. The processor circuitry can evaluate the acute and long-term impact of HFs on physiology and sleep for the user, by tracking changes in physiological functioning (e.g., heart rate (HR) and sleep-wake activity) in association with acute HF events and over time in association with multiple HF events, and also considering the “physiological severity” signature of each HF event. The system has capabilities such as redundancy built in to ensure accurate identification of HF events and other physiological signals that are differentiated from noise and activity unrelated to HF events. The system provides methods for tracking HF events and the impact on the user, and to use the system to automatically trigger an intervention action and monitor effectiveness of the intervention action for the HF event.

While there is variability in the magnitude and signal strength for the detection of HF events and related physiology across different body locations (e.g., wrist, sternum, back of the neck, finger), the predictive data model adapts to signal variations to detect HF events at the body locations. The form factor of the system can include smart sensor circuits, such as rings, wristwatches, jewelry, patches, etc. In some embodiments, a smart ring can be used as a configuration for HF detection as the finger reliably captures changes in blood flow, vasoconstriction/vasodilation, peripheral skin temperature, motion, HR and heart rate variability (HRV), and SC with high signal-to-noise ratio, allowing for constant and stable contact of the electrodes to the body surface.

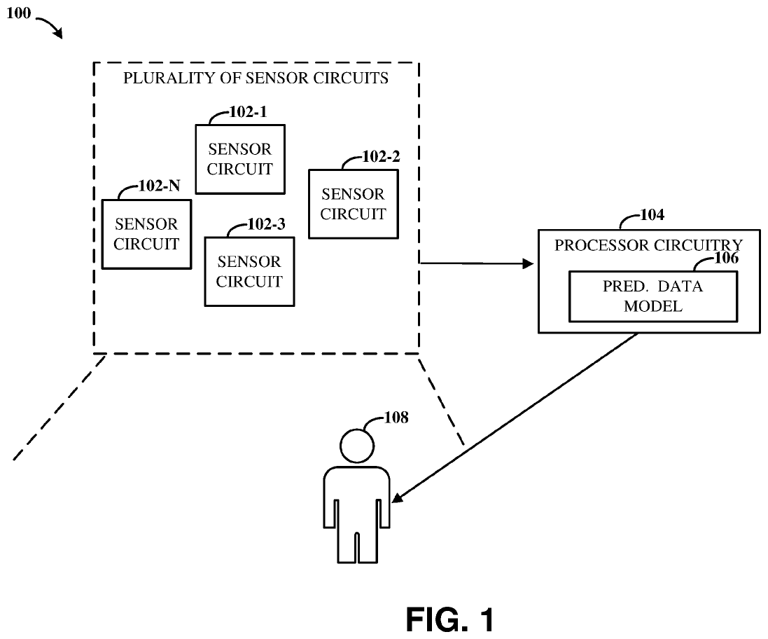

Referring now to the figures, FIG. 1 illustrates an example of a multi-sensor circuit system for hot flash identification, in accordance with various embodiments. The system 100 can be used for managing HF events and in specific embodiments, for managing menopause symptoms. However, embodiments are not limited to a menopause management system and can include a system used to manage HF events from other causes, such as prescription drugs or cancer treatment.

The system 100 includes a plurality of sensor circuits 102-1, 102-2, 102-3, 102-N (herein generally referred to as the “sensor circuits 102” for each of reference) and processor circuitry 104. Each sensor circuit 102 is used to obtain a sensor signal associated with a user 108. In some embodiments, each sensor circuit 102 has a communication circuit for communicating the sensor signal to the processor circuitry 104. The communication circuit can communicate in a wireless or wired manner. As used herein, a “plurality of sensor circuits” is sometimes interchangeable referred to as “multiple sensor circuits” and “multi-sensor circuits”.

The sensor signals obtained from the user 108 can include or be indicative of physiological measurements obtained from the user 108 that are correlated with psychological processes and/or behavior of the user 108. In some embodiments, the sensor circuits 102 can include a wearable physiological sensor, such as a wearable device, that senses the physiological measurements from the user. Example physiological measurements include parameters such as blood pressure, HR, SC, body temperature, and motion data (e.g., from accelerometer and/or global positioning data (GPS)), among other measurements. However, embodiments are not limited to a physiological measurements and can additionally or alternatively include other measurements.

In some embodiments, the system 100 includes two or more sensor circuits selected from a PPG sensor, a SC sensor, a temperature sensor, and a motion sensor. In some embodiments, the system 100 includes at least four sensor circuits including at least one of each of a PPG sensor, a SC sensor, a temperature sensor, and a motion sensor. The plurality of sensor circuits 102 can be located at different body locations of the user 108 or at the same body location. As described above, the different body locations can include a wrist, sternum, back of the neck, and/or finger of the user 108. Use of the sensor signals from the sensor circuits 102 can be used to recover from erroneous sensor measures with a threshold level of tolerance.

The processor circuitry 104 is in communication with the plurality of sensor circuits 102. The processor circuitry 104 can include or otherwise have access to a predictive data model 106. For example, the predictive data model 106 (among other data) can be stored on memory in communication with the processor circuitry 104. The predictive data model 106 can be indicative of a probability of a HF event occurring for the user 108 (e.g., a probability the user will have a HF) at a particular date and time based on a plurality of input parameters, such as the features associated with the sensor signals from the sensor circuits 102. As used herein, a HF event includes or refers to a HF at a particular point in time. A HF can include a plurality of HF events over a range of time (e.g., the HF occurs for five minutes, and a HF event is identified or detected by the system 100 every ten seconds of the five minutes). In other embodiments, a HF can include one HF event. The communication can be one-way or two-way. For example, the processor circuitry 104 can be provided with sensor data (e.g., values) from the sensor circuits 102, which can be digitized and processed. In some embodiments, the sensor data is communicated from the sensor circuits 102 to the processor circuitry 104. In other examples, the processor circuitry 104 reads the sensor data and/or otherwise communicates with the sensor circuits 102, such as sending a message to a sensor circuit for configuration.

The processor circuitry 104 can receive the sensor signals from the sensor circuits 102 and identify a HF event for the user using the predictive data model 106. For example, the processor circuitry 104 can extract features from the plurality of sensor signals obtained by the plurality of sensor circuits 102, align the extracted features to a common time point, and identify a HF event (e.g., occurring or predicted to occur) for the user 108 using the predictive data model 106 based on the aligned extracted features. For example, the features can be aligned to a common time point, with each feature having different windows but the same window center time-point.

The predictive data model 106 can be indicative of probability of the HF event occurring for the user at a date and time. Features can be input to the predictive data model 106 and the probability is output from the predictive data model 106. The predictive data model 106 can include one or more patterns associated with feature sets of the sensor circuits 102, where each feature of a feature set can impact or contribute to the probability of the HF event and/or the user 108 being in a particular psychophysiological state. The feature sets can include an order and timing of the features occurring, among other attributes of the factors, such as amplitude or strength. In some embodiments, the processor circuitry 104 evaluates the user psychophysiological state, and the real-time multi-sensor based HF classification triggers the evaluation of the immediate impact of the HF event (e.g., HF sleep impact), activates real-time interventions (e.g., immersive meditation, cooling solutions), and provides feedback to the users (e.g., communicates a message indicative of the HF event).

The predictive data model 106 can include an artificial intelligence (AI) model or machine learning model (MLM). Various ML frameworks are available from multiple providers which provide open-source ML datasets and tools to enable developers to design, train, validate, and deploy MLMs, such as AI/ML processors. AI/ML processors (sometimes referred to as hardware accelerators (MLAs), or Neural Processing Units (NPUs)) can accelerate processing of MLMs. ML processors are integrated circuits (ASICs) that can have multi-core designs and employ precision processing with optimized dataflow architectures and memory use to accelerate calculation and increase computational throughput when processing MLMs.

For example, extracted feature sets can be input into the predictive data model 106 and used to identify the pattern from the extracted features. Based on the pattern and the input, the predictive data model 106 can output a probability of the HF event. In some embodiments, the output can include and/or be used to select the intervention action to decrease the probability of the HF event and/or decrease the severity of the HF event, such as automatically causing an intervention action to occur.

Each different sensor circuit 102 can be associated with different features that are indicative of a HF event occurring or not. That is, each sensor has its own feature set. The following is a non-limiting example of different feature sets: HR estimation (e.g., Fast Fourier transform (FFT)-based HR) for the PPG sensor, area under the curve (AUC) and HF onset for the SC sensor, average temperature for the temperature sensor, and average movements in x, y, and z dimensions for the motion sensor. Sensor signals can be sampled at different (sampling) rates (e.g., 500 Hz, 20 Hz, 1 Hz), and have an associated time window in which the sensor signals are computed corresponding to the type of features to be extracted. For example, the AUC from the SC sensor can be computed using some context preceding the signal (e.g., time window). In some embodiments, sensor signals are processed in regular time intervals (e.g., one second) and the processor circuitry 104 uses the current sensor signals with prior sensor signals, such as the last 250 seconds of sensor signals.

As noted above, the sensor circuits 102 can be associated with a plurality of different time windows and after extracting the features, the features are time-aligned based on the location of the respective time window. For example, the sensor signals can be obtained by the sensor circuits 102 at or based on the different time windows. The processor circuitry 104 can align the extracted features to a common time point based on the plurality of different time windows associated with the plurality of sensor circuits 102. In some embodiments, the processor circuitry 104 can weigh each of the extracted features based on an impact of the extracted features on the probability of the HF event occurring. For example, the different weights can be dependent on an impact of the feature to a psychophysiological state of the user 108, such as if the user 108 is awake or asleep.

In some embodiments, the predictive data model 106 includes a plurality of sub-models that each indicate the probability of the user having a HF at particular time of the day. Each sub-model is associated with a particular sensor circuit 102, which can have a set of features and associated weights for each feature of the set. The weights can be based on or indicative of how predictive the respective feature is for the user 108 to have a HF in the past or for other users to have a HF (e.g., how accurate of a predictor the parameter is for occurrence of a HF). For example, the weights can be based on or be indicative of an impact or correlation of the features on the probability of a HF occurring.

The following describes two example approaches for combining the information from the sensor circuits 102. The approaches can be referred to as “late fusion” and “early fusion” for ease of reference. In late fusion, the predictive data model 106 includes sub-models associated with each sensor circuit 102 and each sub-model provides a score. Once each sub-model provides a score, a final weight is computed by combining the scores of the sub-models and producing a single score. One advantage of the late fusion approach is that it has lower complexity when adapting the system 100 on the fly. In some instances, the system 100 can adapt the sensor sub-models based on the user inputs in real-time, such as the event marker. In addition, the final fusion weight can be adjusted and reweighted based on the event marker to bias the prediction towards weighting the reliable sensor circuit for the current (e.g., real-time) session.

For example, and in accordance with the late fusion approach, the predictive data model 106 includes a plurality of sub-models, and each of the plurality of sub-models are associated with a respective sensor circuit of the plurality and are each used to provide an output score indicative of the probability of the HF event occurring for the user 108 based on the extracted features from the respective sensor signal obtained by the respective sensor circuit. The processor circuitry 104 can combine the output scores from the plurality of sub-models to identify the HF event (e.g., is the HF occurring (or about to) or not).

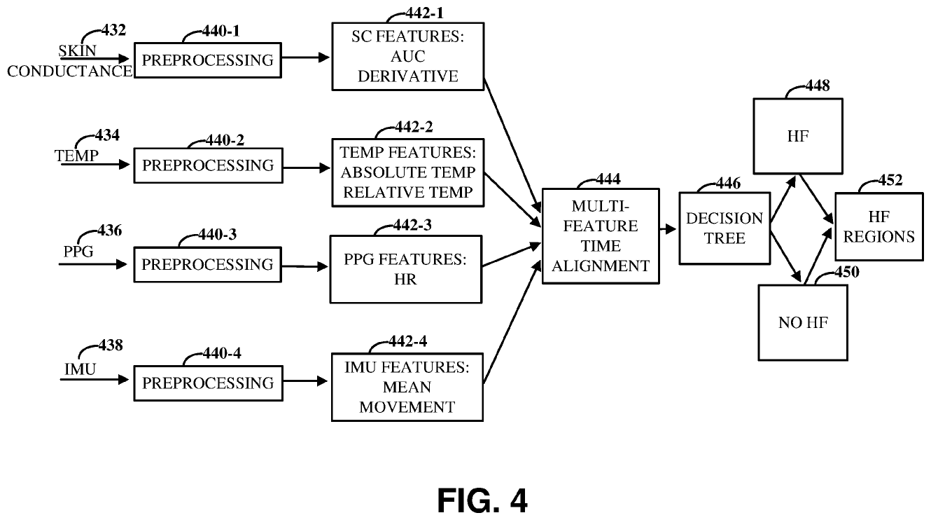

In early fusion, the features are combined to form a vector at each time-point. The vector of combined features is used as input to predictive data model 106 which produces a HF score. A threshold is used to decide whether there is a HF event at the current time-point. In this approach, a decision tree structure can used to combine multiple features. The early fusion approach allows for several decision paths to be used, which adds robustness and redundancy to the decisions. The decisions at each time-point (instantaneous decisions) can be combined by detecting consecutive HF events and converting to time-regions, sometimes referred to as “HF regions”. The advantage of the early fusion approach is tighter feature integration and which allows for higher HF accuracy by better exploiting feature relations. An early fusion process is shown by FIG. 4 below. The decision tree structure can include a binary tree structure. For example, the features can be used by a binary tree structure in successive thresholds (e.g., higher or lower threshold indicate different weights), which are learned from the data. The thresholds can be adjusted manually and/or automatically by the predictive data model.

For example, and in accordance with the early fusion approach, the processor circuitry 104 can combine the extracted features from the plurality of sensor signals into a vector and input the vector to the predictive data model 106 to produce an output score indicative of the probability of a HF event. The processor circuitry 104 can compare the output score to a threshold and identifies the HF event is occurring if the output score is above the threshold and is not occurring if below the threshold for both the early and late fusions.

In some embodiments, the processor circuitry 104 can track HF events over time. For example, the processor circuitry 104 can generate and/or use a decision tree structure to combine the extracted features and to produce an output score based on the combine extracted features. In some embodiments, the processor circuitry 104 can identify whether the HF event is occurring or not at a plurality of time points based on output scores, detect consecutive identified HF events, and convert the consecutive identified HF events into a HF region. A decision tree, as used herein, includes or refers to a data structure that forms part of, or includes the predictive data model 106 that represents different decisions as branches to reach outputs or decisions that are represented as leaves. The decision tree can be used to predict the probability of a HF event based on a plurality of input features extracted from the sensor circuits 102, and can be used to provide fusion of a set of complex rules as multiple paths.

In some embodiments, the processor circuitry 104 can characterize a level or a presence of the HF event based on the extracted features. The level of the HF event can include or be indicative of a severity of the HF for the user 108. For example, based on the magnitude of feature(s) which can include a feature other than or in addition to SC amplitude, such as HR rise, amount of wake time, etc., the processor circuitry 104 can characterize the level of the HF event.

In some embodiments, the processor circuitry 104 can communicate a message indicative of the HF event to the user 108. In some embodiments, the message includes at least one of the identification of the HF event and an intervention action for the HF event. The intervention action can reduce the probability of the HF event occurring and/or reduce a severity of the HF event. In some embodiments, the message is communicated to a computing device with a user interface, and the user interface is used to provide the message to the user 108 to induce the user 108 to perform the intervention action. In other embodiments, the message is communicated to the computing device or an actuator to automatically cause the intervention action to be performed by the computing device or the actuator.

Example intervention actions include activating cooling circuitry (e.g., providing spot cooling to one or more locations on a subject's body or turning on a fan or other cooling device in close proximity to the subject), adjusting a temperature of a room, providing sound or haptic feedback (e.g., playing music), and providing other sensory feedback (e.g., providing a particular smell, video, image, virtual reality), among other interventions.

The processor circuitry 104 can communicate the message indicative of the intervention action in response to the probability of the HF event occurring being outside, such as above, the threshold. The intervention action can be based on past user response to an intervention action by the system 100 and/or other users' response to the intervention action to prevent or mitigate HFs and/or other symptoms. In other embodiments and/or in addition, the intervention action includes a computer-readable instruction that is communicated to another device, such as to the sensor circuits 102 or to actuators, to cooling circuitry, and/or to other devices such as temperature control circuitry (e.g., associated with a heating, ventilation, and air conditioning (HVAC) system). As non-limiting examples, the intervention action can be an instruction provided to an HVAC system to change the temperature of a particular room, an instruction to a user device to provide a notification to the user, such as a smart watch beeping to notify the user of a likely or imminent HF, and/or a display on an application executed by a smartphone which instructs the user on a particular action to take, among other specific actions. As another specific example, the intervention action can include an instruction to activate cooling circuitry to provide cooling to the user 108 in response to the instruction. In various embodiments, the intervention action can include an instruction communicated back to one of sensor circuits 102-1, which causes the sensor circuit 102-1 to adjust the measurement (e.g., increase or decrease the sensitivity and/or number of measurements). As may be appreciated, embodiments are not limited to a single intervention action and multiple actions can be triggered by the system 100.

In some embodiments, the processor circuitry 104 is configured to identify the HF event in real-time. The message can be used to automatically trigger intervention actions for HFs. As described above, the message can be sent to a user device to communicate to the user 108 or to an actuator (or other device) which performs the intervention action.

The processor circuitry 104 can receive feedback data in response to the communicated message. The feedback data can be indicative of at least one of a user confirmation of the HF event, a user denial of the HF event, and a severity of the HF event. In some embodiments, the feedback data can include verification of an occurrence of a HF at a particular date and time, body location of the HF, and/or a severity or impact of the HF on the user 108. In some embodiments, the feedback data can be used to identify changes in HF patterns over time. The feedback data can be manually entered by the user 108 to the system 100, such as using a user interface of a connected device, and/or determined or inferred from sensor signals obtained by the sensor circuits 102. In various embodiments, the feedback data can be used to revise the predictive data model 106, such as revising adjusted weights for extracted features as further described below. In various embodiments, the feedback data can include expert scores of HF events. However, examples are not so limited and the feedback data can include other types of feedback data such as user scores, sensor data, and other data.

In some embodiments, the predictive data model 106 can be dynamically updated over time. For example, the processor circuitry 104 revises the predictive data model 106 over time using the feedback data that is indicative of experienced HF events for the user 108 and/or other users, and/or additional input data. The update can include adjusting the weights of different input parameters based on the experienced HF events and/or impact of intervention actions. As a specific example, over time, the user 108 can experience a change in HF occurrences that results in additional HFs at night. In another example and/or in addition, the feedback data can be indicative of specific information of the HF event, such as a body location of the HF, a severity or impact of the HF, and/or intervention actions that are believed to result in mitigation of the HF and which can be used to adjust the intervention actions for the user 108 and/or adjust weights for features. Different users can experience relief, mitigation, and/or prevention of HF events using different intervention actions. As a specific example, the particular user 108 may have relief from HFs at different times of the day in response to cooling provided to different locations of the body.

In various embodiments, the predictive data model 106 is dynamically updated over time based on the severity or impact of past HF events and/or at particular times of the day. The severity or impact can be a scaled parameter, such as a user provided number of between 1-10, with 10 being the most severe or highest impact and 1 being the least. Other types of numerical scales can be used, such as 0-100 or one to five stars. As specific examples, a HF at night that wakes up the user 108 can have a higher impact than a HF that does not wake up the user 108. As another example, a HF while the user 108 is in a meeting can have a higher impact than when the user 108 is at home.

Based on the predictive data model 106, which is dynamically updated over time, the system 100 can be used to predict occurrence of a HF event and to anticipate an imminent HF. For an imminent HF, the system 100 can proactively mitigate or prevent the HF using intervention actions. For a predicted HF, in some specific embodiments, the system 100 makes suggestions to the user 108, proactively mitigates or prevents the HF, and/or increases the amount or sensitivity of sensor signals in order to more accurately detect an imminent HF and which can allow for reduced power consumption of the sensor circuits 102 (which can be in a lower power consumption mode prior to the predicted HF event).

In some embodiments, the processor circuitry 104 generates the predictive data model 106 and stores the predictive data model 106, such as in a coupled memory circuitry. In other embodiments, the processor circuitry 104 receives the predictive data model 106 and stores the predictive data model 106 in a coupled memory circuitry. As previously described, the predictive data model 106 can include a MLM which is trained using input data.

Generating the predictive data model 106 can includes receiving input feature sets and associated past HF events for the user 108 or other users, identifying different patterns or correlation of occurred HFs and the input feature sets, and, based on the patterns, identifying predictive probabilities of the user 108 having a HF at dates and times in response to different feature sets. In some embodiments, additional data can be input. The input data can include lifestyle and other HF factors for the particular user 108 and/or other users and the physiological measurement(s) from the sensor circuits 102. The processor circuitry 104 receives the plurality of input data and uses the same to generate the predictive data model 106.

The predictive data model 106 can be applied online or off-line and can be adapted to different sensor configurations, including the use of dry or wet electrodes, contact and non-contact sensors and from monitoring of sensor signals from different body locations (e.g., wrist, back of the neck, finger). The system 100 reads input data streaming from multiple sensor circuits 102.

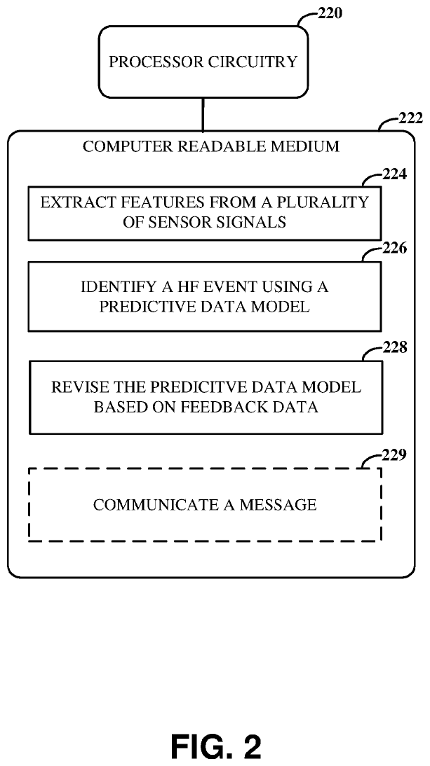

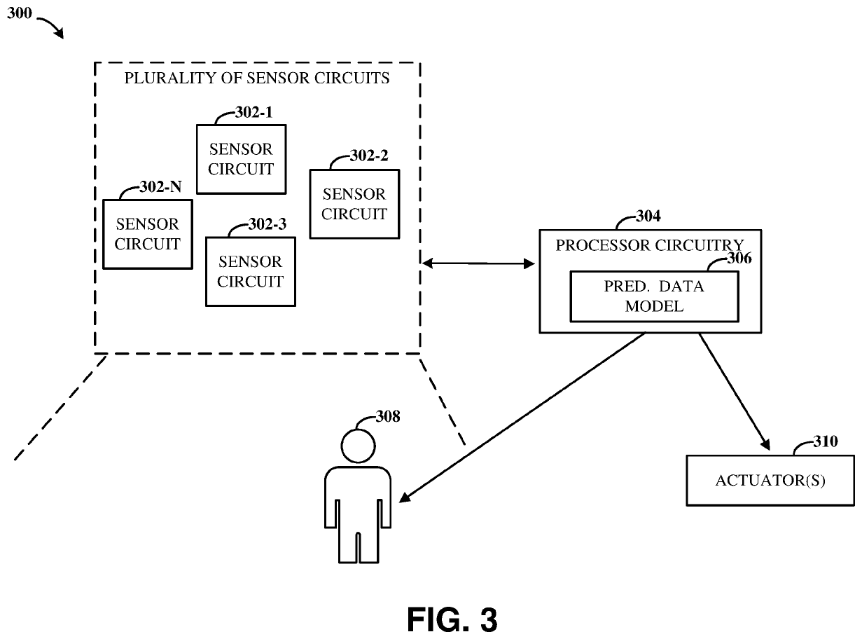

FIG. 2 illustrates an example computing device including non-transitory computer-readable medium storing executable code, in accordance with the present disclosure. The processor circuitry, in accordance with examples herein, can include the processor circuitry 104, 304, illustrated by FIGS. 1 and 3.

In some examples, the processor circuitry 220 can form part of a computing device. The computing device includes the processor circuitry 220 and computer readable medium 222 storing a set of instructions 224, 226, 228, 229. Although embodiments are not so limited and the processor circuitry 220 and computer readable medium 222 can form part of distributed computing devices, which are in communication.

The computer readable medium 222 can, for example, include read-only memory (ROM), random-access memory (RAM), electrically erasable programmable read-only memory (EEPROM), Flash memory, a solid state drive, and/or discrete data register sets.

At 224, the processor circuitry 220 can execute instructions to extract features from a plurality of sensor signals associated with a user, the plurality of sensor signals being obtained by a plurality of sensor circuits. In some embodiments, the processor circuitry 220 can align the extracted features to a common time point based on a plurality of different time windows of the plurality of sensor circuits used to obtain the plurality of sensor signals.

At 226, the processor circuitry 220 can execute instructions to identify a HF event for the user using a predictive data model indicative of a probability of the HF event occurring for the user at a date and time based on the extracted features. At 228, the processor circuitry 220 can execute instructions to revise the predictive data model based on feedback data indicative of an impact of the HF event on the user. The feedback data can be received from devices external to the processor circuitry 220, such as from respective sensor circuits, actuators, or from other user devices. The feedback data can include at least one of a user confirmation of the HF event, a user denial of the HF event, a severity of the HF event (e.g., user provided or based on sensor signals), and an impact of an intervention action on the HF event.

In some embodiments, each extracted feature is associated with a weight indicative of an impact of the respective feature on the probability of the HF event. In some such embodiments, the processor circuitry 220 can revise the predictive data model by adjusting a first weight associated with a first feature of the extracted features, adjusting the first weight and a second weight associated with the first feature for different psychophysiological states of the user, and/or adjusting an intervention action for additional HF events. Although the above-example describes adjusting a first weight, examples are not so limited and can include a plurality of weights being adjusted, such as two, three or more weights. In some examples, all of the weights for the plurality of features are adjusted. For example, during different psychophysiological states, such as the user being awake or asleep, respective features can be more or less relevant to the probability of a HF occurring. The weights can be automatically and/or manually adjusted.

In some embodiments, at 229, the processor circuitry 220 communicates a message indicative of the HF event to the user, wherein the message indicates least one of an occurrence of the HF event, a prediction of the occurrence of the HF event, and an intervention action for the HF event. The intervention action can be based on past responses of the user to the intervention action and/or other users to the intervention action, and that can include mitigation and/or prevention of a HF and/or other menopause symptoms. For example, past responses of other users that are demographically similar to the user can be used to select an intervention action, and/or can be updated over time based on responses of the user.

In various embodiments, the communicated data includes an instruction that activates an actuator. As an example, cooling circuitry can be worn by the user. In response to the activation, the cooling circuitry provides cooling to the user to mitigate or prevent an imminent or occurring HF event. The processor circuitry 220 can generate another instruction to deactivate the cooling circuitry based on additional sensor signals from the plurality of sensor circuits indicating no HF event and/or that the HF is over. Embodiments are not limited to activating cooling circuitry and can include other types of actuators, as described herein, and/or providing a message to the user via a user interface to initiate the intervention action.

FIG. 3 illustrates another example multi-sensor circuit system for HF identification, in accordance with various embodiments. The system 300 includes a plurality of sensor circuits 302-1, 302-2, 302-3, 302-N (herein generally referred to as the “sensor circuits 302” for ease of reference) and processor circuitry 304 as previously described in connection with FIG. 1. In some embodiments, the system 300 further includes one or more actuators 310, such as cooling circuitry.

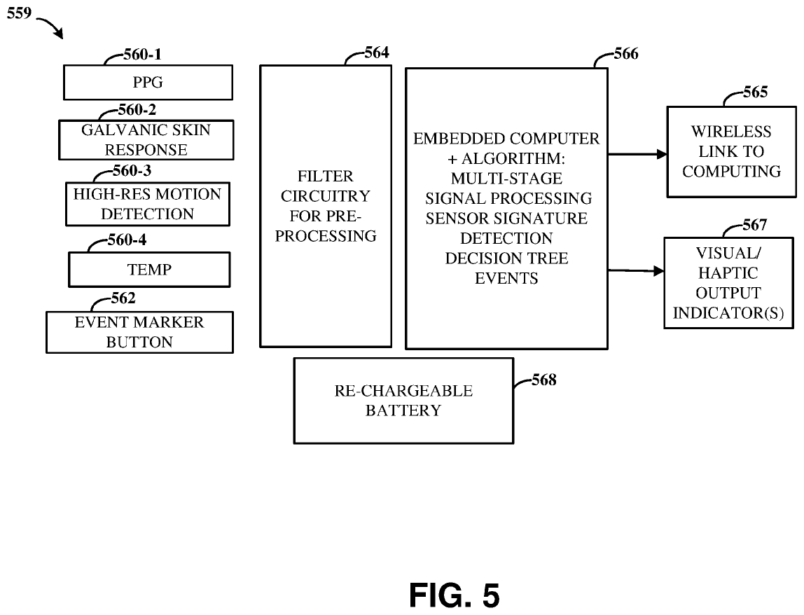

Similarly to that described in connection with FIG. 1, the sensor circuits 302 obtain a plurality of different types of sensor signals from the user 308 and communicates the sensor signals to the processor circuitry 304. In some embodiments, one or more of the sensor circuits 302 includes a wearable physiological sensor to sense a sensor signal from the user 308 that is indicative of a physiological measurement. The sensor circuits 302 can be wirelessly linked to a smartphone or other device executing an application. As further illustrated by FIG. 5, the system 300 can additionally include a component to communicate with the user 308. Any of the available types could be used to indicate detection (e.g., confirmation feedback) or to indicate an intervention action (e.g., suggested response by the user 308) or trigger the intervention action by a device as described above (e.g., initiating a cooling sensation). The system hardware can provide this via a multi-colored LED, a vibration from a haptic device or a suite of low sounds. Any of these can be embedded in a wearable device or be on a third wireless device, or in some use cases, a linked smartphone can be the actuator for visual, vibratory or audio response.

As with described above in connection with FIG. 1, the processor circuitry 304 can extract features from the plurality of sensor signals and align the extracted features to a common time point based on the plurality of different time windows associated with the plurality of sensor circuits 302.

The processor circuitry 304 can track a psychophysiological state of the user 308 based on the aligned extracted features. The tracked psychophysiological state can include or be associated with a sleep state and/or an awake state of the user 308. Tracking the psychophysiological state can be used to adjust weights applied to different features extracted from the sensor signals and/or to determine an impact of the HF events on the user 308. As an example, the processor circuitry 304 can track the amount of time in a sleep state and in an awake state, and calculate an amount of time in the awake state that is associated with or caused by a HF event or a plurality of HF events. Different features can indicate the user 308 is in a particular psychophysiological state, such as lack of movement and HR below a threshold indicating a sleep state. Some features can indicate a transition between states, such as a change in the amount of movement or increase in HR from the HR below the threshold.

The processor circuitry 304 can track a plurality of HF events for the user 308 using the predictive data model 306 indicative of the probability of a HF occurring for the user 308 at a date and time based on the aligned extracted features and the tracked psychophysiological state of the user 308. As described above, the predictive data model 306 includes weights for each of the extracted features and for each of the different psychophysiological states, each weight being associated with the probability of the HF occurring at the date and time.

The processor circuitry 304 can communicate a message (e.g., one or more messages) indicative of the plurality of HF events to the user 308. The message can be indirectly communicated to the user 308, such as being sent to an actuator to automatically cause an intervention action. In other embodiments or in addition, the message can be sent to the user 308 via a separate device and a user interface.

In some embodiments, the processor circuitry 304 can revise the predictive data model 306 based on feedback data indicative of an impact of the plurality of HF events on the user 308. For example, the HF events can tracked over time and intervention actions can be adapted based on the feedback data received from the user 308 and/or from the sensor circuits 302. The feedback data can be indicative of the severity of the HF event, the impact of intervention action on the HF event, and/or if the intervention action occurred. As a specific example, extracted features can indicate whether or not the intervention action modulated the physiological response of the user 308. As another example, the user 308 can manually rate the severity of the HF event and/or the impact of the intervention action. The revision can include adjusting weights for the extracted features as associated with the tracked psychophysiological state (e.g., different sensor circuits 302 can be better or worse for user and/or better at classifying in different states).

The HF events and/or impact of intervention actions can be tracked to evaluate short and longer changes in physiological functioning as associated with HF events. For example, HR and sleep-wake activity and respective associated with HF events can be tracked. Similarly, the severity of HF events and trends over time can be tracked.

In various embodiments, the system 300 can include or form part of a computer program or application that can run on a smartphone, a tablet, a desk computer, a laptop, smart watch, exercise tracker, or other independent device where the computer program or application that can run on a smartphone, a tablet, a desk computer, a laptop, smart watch, exercise tracker, or other independent device that can be contained within a wearable device or which is in data communication with a wearable device.

In some embodiments, the system 300 can include or be in communication with one or more actuators 310, such as those previously described. The actuator 310 can be in communication with the processor circuitry 304 directly or through a separate device, such as a smart device, computer, or so forth. The actuator 310 can includes circuitry that provides an intervention action (e.g., mitigating output) in response to the HF event. In some embodiments, the one or more actuators 310 can include cooling circuitry as previously described. In some embodiments, the cooling circuitry can be integrated with the system 300 into one application or device that can be used to mitigate the effects of HFs. Embodiments are not so limited and can include other types of actuators 310, as previously described. In some embodiments, the devices can be in either wired or wireless communication with the device on which the system 300 is running.

FIG. 4 illustrates an example of a multi-sensor signal processing, sensor-wise feature extraction, combination, and final HF region(s) extraction, in accordance with various embodiments. The system illustrated by FIG. 4 can include a plurality of sensor circuits 432, 434, 436, 438, including a SC sensor 432, a temperature sensor 434, a PPG sensor 436, and an IMU sensor 438.

Sensor signals from the sensor circuits 432, 434, 436, 438 can be processed independently. Processor circuitry can process the data to make it more suitable for analysis. Data from each sensor signal can be processed differently. For example, processing of data from the sensor circuits 432, 434, 436, 438 includes but is not limited to low pass filtering, averaging, smoothing, and so on.

The processor circuitry can pre-process sensor signals, at 440-1, 440-2, 440-3, 440-4, for artifact removal, and multi-features extraction, including gravity, SC slope and AUC, PPG mean absolute energy, and PPG-derived HR. After the pre-processing, the pre-processed sensor signals can be independently used to compute feature sets 442-1, 442-2, 442-3, 442-4 for each sensor circuit 432, 434, 436, 438.

As previously described, each sensor circuit 432, 434, 436, 438 can include its own set of features. The following are non-limiting examples of feature sets for each sensor circuit 432, 434, 436, 438: AUC and HF onset for the SC sensor 432, average temperature for the temperature sensor circuit 434, HR estimation for the PPG sensor 436, and mean movement for the IMU sensor 438. Sensor signals can be sampled at different sampling rates (e.g., 500 Hz, 20 Hz, 1 Hz), and have an associated time window in which the signals are computed corresponding to the type of features to be extracted. For example, the AUC from SC sensor 432 is computed using some context preceding the signal window. The features are then time-aligned based on the location of the time window.

As previously described, the features can be time-aligned, at 444, using a late fusion approach or an early fusion approach, the details of which are not repeated. FIG. 4 illustrates an example early fusion approach in which the features are combined to form a vector at each time point, and to produce a HF score. In such embodiments, a decision tree structure can be used to combine the features, at 446, and to determine if there is a HF event, at 448, or no HF event, at 450, at each point in time. The decisions at each time-point (instantaneous decisions) are combined by detecting consecutive HF detections and converting to HF regions, at 452.

In some embodiments, the signal inputs are processed to obtain a representation that allows the analysis module to learn the pattern with respect to the HF event occurrence for a particular user, and which can include use of different machine learning processes. Different ML processes can be incorporated in the predictive data model depending on the input sensor signals.

Based on the “gold standard” measure of HF (e.g., SC signal) and user self-report inputs about HF occurrence and severity, the features of the predictive data model can be optimized by minimizing a cost function of each feature (e.g., for an early fusion) and/or of the sub-models for a late fusion. The cost function is a function that maps the model predicted probability onto a real number intuitively representing some “cost” associated with the predicted probability value.

The input sensor signals can include raw sensor signals or extracted features of raw sensor signal. Extracted features are temporal and/or spectral features representing the sensor signals and their specific time pattern, variabilities and frequency content. Temporal features can include statistical measures, such as mean, variance, and higher order statistics of the input data in a time frame. Spectral features can be extracted using Fourier transform. Spectral features in one example can be spectral moments, spectral power fractions, spectral power peaks, and spectral power ratios. The features can also be extracted after applying an appropriate transform that facilitates the understanding of the input pattern such as wavelet transform. Features can also include parameters of a model best representing the data in a specific time window. Since the dimensions of the input patterns can be very high, statistical methods such as principal component analysis or linear component analysis can be used to transform the features into a lower dimension subspace where a more precise and efficient representation of the input patterns is achieved.

Although FIG. 4 illustrates an early fusion and/or a decision tree, examples are not so limited and can include a late fusion. For example, in various embodiments, the predictive data model can include a plurality of sub-models. Each sub-model can be associated with a different sensor circuit and provides associations between the inputs and the outputs which are current and/or future probabilities of HF occurrence. For example, the output of the sub-models associated with each category of inputs (e.g., feature set of a respective sensor circuit) can be fused to form a final probability P of a HF event occurrence. A simple example is a weighted summation of the output probabilities of the sub-models. To find the weights, methods such as regression analysis can be applied. Because the final probability may not be obtained by a linear weighted summation of the sub-models outputs, more advance ML methods can be used. An example is a ML that accepts the output probabilities of the sub-models and outputs the final probability. Assuming that there are k categories of inputs, the system accepts k inputs (k probabilities of hot flash occurrence based on the input type) and output a single value probability P of HF event occurrence.

Another example is the logistic regression model which defines a linear decision boundary between the training samples associated with a HF occurrence and those that are not. A more complex model can be built when there is a more complex or non-linear relationship between the inputs and output. A deep neural network such as a multi-layer perceptron (MLP) can be used for this purpose.

To calculate the optimum network parameters (weights and biases in the case of neural networks), an optimization operation, such as backpropagation, can be used which can be done in batch or incremental styles. In the batch mode, all available training data are provided to the network to calculate the optimum parameters, while in the incremental style, the parameters are updated each time a training sample is presented to the network.

The optimization of the model parameters is done by minimizing a cost function. An example of for the cost function is the cross-entropy error which the system defines between the estimated HF probabilities and the “true” HF distribution. Given a dataset of N training samples the cross-entropy cost function is defined as follows:

$$J = - {\sum }_{i = 1}^{N} {t}_{i} log ( {y}_{i} ) + ( 1 - {t}_{i} ) log ( 1 - {y}_{i} )$$

where ti is the true HF probability for training sample i that could be either 0 or 1, Yi is the predicted probability which can take any value between O and 1. Minimizing the negative log likelihood cost function is equivalent to maximizing the likelihood of the correct probability.

During training, the cost function is minimized by tuning the model parameters so that the inputs corresponding to a HF event occurrence results in an output probability of close to 1 and inputs that are not associated with the occurrence of a HF results in an output probability of close to 0.

The built model, when fed by new inputs, outputs the probability of HF event occurrence which can vary from 0 to 1. The model is updated over time based on the new user inputs and/or feedback data, and sensor signals regarding the HF occurrence and its severity.

Other MLM that can be used for this purpose can include naive Bayes, probabilistic decision tree and probabilistic support vector machines classifiers. Other structures of neural networks can also be incorporated such as recurrent neural networks, radial basis neural networks, etc.

The above described systems and computer-readable instructions can be used to track HF events and impact of HF events for a user and to mitigate the effects of the HFs by generating a predictive data model which is dynamically updated over time using sensor signals from multiple sensor circuits. Based on the dynamic predictive data model, the system is used to predict occurrence of a HF, to anticipate an imminent HF, and to mitigate symptoms caused by HF and/or other symptoms of menopause. The systems can be implemented as multiple devices in communication and/or as single wearable device including the multiple sensor circuits.

FIG. 5 illustrates an example of a device forming at least part of a system, in accordance with various embodiments. In some embodiments, the system includes a wearable sensor circuits linked to a computer or smart phone, which is the device 559. The computer or smart phone can receive from a plurality of inputs.

The system includes but is not limited to a multi-sensor circuit array, such as sensor circuits to sense signals associated with PPG 560-1, GSR 560-2, skin potential, temperature 560-4, IMU 560-3, sweat-rate sensors, and event marker 562 (e.g., a button the user can press to indicate a HF onset), among other inputs. In some embodiments, the sensor circuits are fully integrated on the device 559, and in other example, can be in communication with the device 559. The sensor signals becomes part of the recorded data to increase confidence in recognizing HF signatures for the individual for bio-behavioral measurements. The system further includes the device 559 which can be referred to as local computing module (including wireless connectivity, microcontroller, and portable battery 568) and can further include a local user interface (e.g., red green blue (RGB)/light emitting diode (LED), audio, haptic stimulation), a mobile device application (or tablet, personal computer (PC)), and optionally, can be in communication with a cloud computing system.

The different inputs can be pre-processed, as previously described by FIG. 4, by filter circuitry 564 and processor circuitry 566 that can pre-process sensor signals and detect a HF event using a predictive data model. In some embodiments, the device 559 includes a smartphone in communication with the wearable sensor circuits. The system and/or device 559 can include an additional component provides feedback data from the user, such as from the sensor circuit or another wearable device. Any of available type can be used to indicate detection of HF event (e.g., confirmation feedback), to indicate an intervention action (e.g., suggested response by the user), and/or to trigger an intervention action by actuators as mentioned above (e.g., initiating a cooling sensation). In some embodiments, the device 559 can provide the intervention action via visual or haptic output 567 (e.g., a multi-colored LED, a vibration from a haptic device or a suite of low sounds) or a wireless communication link 565 to another device and/or circuit. Any of these can be embedded in the device 559 or be on a third (smart) wireless device, or in some use cases, the linked smartphone can be the actuator source (for visual, vibratory or audio response).

The predictive data model can exist in part or in total within the firmware of the device 559, such as on an internal processor. Preprocessing of the sensor signals can occur inside in a mix of circuitry/firmware for things such as noise filtering, offset removal, trend tracking, down-sampling of the data and pre-selecting high-likelihood signature events from sensors. Potential benefits mean faster detection, redundancy in analysis, and little or no computing overhead in peripheral phone and/or PC. The peripheral devices can become long-term data collectors and which can minimize user interaction with a user interface.

Associated hardware elements can be suitable primary sensors, suitable digital signal processing (DSP) and amplifiers that are quiet, high-sensitivity and small. These can be coupled with a moderately fast microcontroller, such as a 32-bit ARM, to sample the sensor signals and perform the data processing. As further described below, a real-time response can be provided.

Example systems, devices, and methods provide a multi-sensor circuit approach for HF identification based on the detected physiology of HFs (rises in SC, vasodilation, tachycardia, increase in skin temperature) and user behaviors reflected in sensor readings (e.g., motion associated with HFs, drop in skin temperature associated with HFs occurring at night due to removal of sheets in addition to dissipation of sweat) in response to HFs events. This approach uses information from different sensor circuits and from different time points. For example, the system, device, and/or methods use changes of temperature and SC over time as features to predict HF events. Having multiple sensor circuits in combination with multipath decision rules described above allows the system to recover from erroneous sensor measures and provide correct results with some tolerance level.

The example systems, devices, and methods allow for HFs severity characterization. Multi-sensor circuit approach for HF severity determination uses a multitude of features (alone or in combination) in addition to or other than SC amplitude (e.g., magnitude of HR rise, amount of wake associated to HFs occurring at night, etc.) to determine the severity of the HF events.

The example systems, devices, and methods can include tree-based decision approaches, which allows for a multiple path tree-based decision rather than a linear decision path. This allows for the fusion of more complex rules such as if the temperature rises and SC rises or if temperature drops and skin conductance rises very rapidly and mark the signal region as HFs. This allows for more complex rules to be evaluated than a single linear set of rules.

The example systems, devices, and methods allow for real-time HFs detection. Another aspect of the methods is the ability to have a causal system that uses only past information. This is done by processing the signal in regular time intervals (e.g., 1 second), and using the current signal and its immediate history (e.g., the last 250 seconds). This approach allows for the system to work in real-time and provide instant feedback which can lead to immediate intervention action (s) (e.g., a tangible physical actuator response), such as adjusting the room temperature, triggering of a cooling therapeutic or cooling sensation to the skin, suggesting a behavioral intervention (e.g., slow breathing).

Associated with the HF identification methods described above, are methods for mitigating HF experience. More specifically, disclosed are methods and mitigation strategies, for novel immersive experience for inducing psychophysiological relief in a user experiencing HF. Accordingly, in some embodiments, the device, systems, and/or methods described herein can additionally or alternatively be directed to mitigation strategies for HFs.

The mitigation strategy can deliver a personalized adaptive mind-body immersive experience to acutely induce specific psychophysiological responses (e.g., relaxation, anxiety reduction, improved mood) and sensory experiences (e.g., feeling of coldness), targeting the specific state of discomfort in a user experiencing HFs. A combination of techniques to achieve immersion (e.g., binaural sounds, virtual reality, haptic feedbacks), psychophysiological responses to the immersion (e.g., mood changes) and specific sensory experiences (e.g., induced feeling of coldness) can be used.

A number of embodiments include a mitigation strategy that includes an immersive solution to induce psychophysiological relief for a user at the time of experiencing a HF. In response to an identified HF event, mitigation strategy can include one or more intervention actions that can be delivered via mobile platforms and/or rely on inputs/outputs (e.g., respiration, HR, skin conductance, motion) from additional devices (e.g., multi-sensor wearable fitness/sleep trackers). A combination of techniques, sound effects to create an immersive dynamic soundscape (e.g., an auditory scene obtained by placing sounds in space and time with the goal of increasing the user presence in the virtual scene using a combination of techniques like binaural recordings, looming effects, etc.), haptic feedbacks, etc., can be activated based on the psychophysiological state of the user. An example is a twenty minute immersive sensory meditation (e.g., guided visual imagery in which a person is walking through heavy snow in a forest with the wind blowing around and sound effects paired with mediation script) aiming at improving relaxation and inducing a feeling of cold. The disclosed system, mitigation strategy, and/or intervention strategy can be adaptive. For example, the system and/or device can monitor the user's physiology in real-time (e.g., via high frequency HRV analysis by real-time processing of a PPG signal from a smart wristband) and the system and/or device adapts the meditation experience if the chosen HRV index reflecting relaxation does not reach a desired level within a desired time. Similarly, the emergence of a HF can be tracked in real-time, and the meditation experience adapted as a user goes from sweating profusely during the HF to shivering after the HF is ended. Changing the script online (e.g., adding nature elements such as a mountain or a lake) or enhancing the soundscape (e.g., adding binaural sounds of the wind moving through the trees, etc.) are among the several strategies that can be used to modulate the meditation experience (meditation-related changes in the user psychophysiological state).

In some embodiments, the mind-body immersive solution can be used when a user is experiencing a HF and is seeking immediate relief. The intervention action and/or strategy can be used to induce feelings of coldness, for example using an immersive soundscape (e.g., binaural sounds of wind blowing, walking through a snow path) paired via guided meditation, to achieve feeling of immersion in a cold environment, and thus mitigate the discomfort associated to the HF. External cooling (e.g., cooling device) can also use in combination with the immersive soundscape. Pace breathing can also be added to directly target relaxation in addition to coldness.

In various embodiments, the example system, methods, and devices can induce immediate psychophysiological relief for a user at the time of experiencing a HF, such as via an immersive solution. The intervention action and/or mitigation strategy can aim at inducing acute changes in a psychological and physiological state of a user by immersing the user in specific environments (e.g., immersive audio tracks, immersive audio tracks plots multi-sensor stimulation/feedback, virtual reality navigation experiences) designed to elicit specific psychological and physiological reactions (e.g., meditation script involving winter themes and winter-related audio effects to induce feelings of coldness).

The immersion solution can be achieved via virtual reality and/or using a designed immersive soundscape, an auditory scene made up of the sounds of a space which can dynamically change over time. Other senses (e.g., smell, tactile) can be also included to achieve a further level of immersion. Immersion can be achieved without technology mediated tools (e.g., visual imagery). Immersion leads to “presence”, e.g., the feeling of being in the virtual environment, and presence may mediate the effect of a virtual environment on the user's physiological perception (e.g., a “positive environment” leads to positive feelings, a “cold environment” can produce cold feelings). Different techniques can be used to obtain psychophysiological changes via audio immersion. For example, auditory frisson can be described as the feeling of coldness in the absence of a physical cold stimulus and can be induced by binaural sounds moving into the users' peri-personal space as described in S. Honda, et al., “Proximal Binaural Sound Can Induce Subjective Frission”, Front Psychol., 2020 Mar. 3, which is herein incorporated by reference in its entirety for its teaching. Features of the audio stimuli (e.g., loudness, sharpness) can affect the frisson experience. Similarly, the autonomous sensory meridian response (ASMR) can be described as a tingling sensation in the body created by auditory stimuli or trigger words (e.g., whispering), and is followed by a state of relaxation.

Specifically related to HFs, meditation or hypnosis using personal imagery associated with coldness can be effective for HFs management as described in accordance with G. Elkins, et al., “Preferences For Hypnotic Imagery for Hot-Flash Reduction: A Brief Communication”, Int J Clin Exp Hypn, 2010 July, 58 (3):345-9 and G. Elkins, et al., “Randomized Trial of a Hypnosis Intervention for Treatment of Hot Flashes Among Breast Cancer Survivors”, J Clin Oncol., 2008 Nov. 1, 26(31), 5022. Other behavioral techniques like pace breathing can also be effective for HF mitigation and can be incorporated in an immersive solution, such as described in connection with R. Sood, et al., “Paced Breathing Compared with Usual Breathing for Hot Flashes”, Menopause, 2013 February, 20(2): 178-94, and Green et al. “The Cognitive Behavioral Workbook for Menopause: A Step-By-Step Program for Overcoming Hot Flashes, Mood Swings, Insomnia, Anxiety, Depression, and Other Systems, 2012, which can be incorporated in the immersive solution and each of which are herein incorporated by reference in their entirety for their teaching. Tactile cooling can also be incorporated into our immersive solution, for example, with spot cooling (e.g., with a cold pack, cold device, fan, cooling spray) applied to specific body areas with high thermal sensitivity (e.g., upper back), and in a way that complements the immersive imagery to potentially induce a greater sense of immersion and “presence”. Other techniques may be used, such as described in Carmody, et al. “A Pilot Study of Mindfulness-based Stress Reduction for Hot Flashes”, Menopause, September-October 2016, 13(5), 760-9, which is incorporated herein in its entirety for its teaching.

As a specific example, an intervention strategy or immersive solution can include a meditation structure that guides a user. The user is first guided through an introduction to the mediation, and then guided to imagine walking in the snow, which can be accompanied by binary sounds of a person stepping through the snow, binary wind sounds and/or effects. The user is then guided to imagine approaching an icy lake, and other sound effects are paired with the user experience (e.g., braking ice). The user is further guided to autonomic relaxation, such as being guided to slow and pace their breathing through a breathing exercise while still immersed in the ad-hoc designed winter immersive soundscape. The user is then guided to imagine diving into the icy lake, accompanied by binary sounds of waves and underwater sounds, and other effects. The meditation structure can be designed to enhance the immersion and induce cold feelings. Paced breathing can be used to mitigate HF events and/or mitigate a potency of the HF event, such as described above.

The intervention action(s) and/or mitigation strategy can be delivered in an open-loop mode (fixed intervention) or in a close-loop modality (adaptive), e.g., the intervention can be modulated by the users' physiological responses to elements/aspects of the immersive experience or by the perceived impressions. The intervention action(s) can elicit different psychophysiological changes (e.g., relaxation, mood changes) and/or sensory experience (e.g., feeling of coldness). For example, if a user following a guided meditation script reaches a deep level of relaxation in correspondence with a certain scenario/element of the script (e.g., at minute 3:45 of the script the user moves away from a forest and reaches a lake), the adaptive intervention action can reinforce elements of that scenario to further promote relaxation rather than playing the whole track. In the adaptive version, the experience of the user can be different and dependent on the real-time responses to the intervention.

The intervention action(s) can be delivered, and data can be gathered via mobile platforms, or it can also involve or be interfaced with data inputs/outputs from external devices (e.g., multi-sensor circuit wearable fitness/sleep trackers, cooling devices) as those described above.

The system, methods, and devices can mitigate a specific state of discomfort by delivering a personalized immersive experience able to induce specific psychophysiological responses to target that specific state of discomfort. For example, a woman is experiencing a HF and is seeking immediate relief. An intervention action can be designed to induce feelings of coldness and thus mitigate the discomfort associated with the HF. While this intervention action can target HF events when the HF events occur (acutely), it can also be incorporated into more long-term mind-body therapies for HF management, such as cognitive behavioral therapy (CBT). The system can personalize the intervention action(s) further to an individual user to potentially reduce the likelihood of a HF from even happening. For example, if a woman is aware of personal HF triggers (e.g. specific time of day), she can use the intervention in advance of that trigger, which might prevent the HF from happening, or minimize its potency.

### More Detailed/Experimental Embodiments

Embodiments in accordance with the present disclosure include systems, devices and methods involving identification and/or management of HF events or, in specific embodiments, management of menopause symptoms for one or more users. The following provide specific example uses of the above-described systems and not intended to be limiting.

In various experimental embodiments, HF events are tracked using a multi-sensor circuit approach to identify HF events using multi-feature integration from a consumer grade skin conductor sensor (SC), a temperature sensor (T), a motion sensor (M), and PPG sensor (PPG) that are placed on the wrist. The wrist can be a useful location as many different consumer wearable devices are located at the wrist, such as smartwatches. Expert evaluation of sternum SC fluctuation was used as a gold standard reference for comparison. Sensor performance for HF identification was additionally evaluated when the user is a sleep state verses an awake state and based on sensor loss of contact or faulty sensors.

In accordance with embodiments, three women (age, mean±standard deviation (SD): 55.6±0.6 years) who reported having daily HFs participating in an around 12 hour lab-based study, that encompassed overnight. A total of 27 HFs were recorded from the women. The women were free from major mental and medical condition, had undergone natural menopause, and none are of a hallmark of the menopause transition. HFs were characterized by peripheral vasodilated and sweating, lasting one to five minutes, and which can occur hourly and/or dialing.

The current gold standard for measuring HF is the expert evaluation of sudden increase (2 μS/30 s) in sternal SC recorded via laboratory or ambulatory research-grade devices. A predictive data model was used. Prior predictive data models are based on sternum SC signal processing (e.g., using fixed SC threshold, pattern recognition techniques, neural networks, template matching). The predictive data model used considers a magnitude of other physiological changes that accompany HF, including increases in HR and increases in skin temperature. Different factors have variable impact on the probability of HF depending on if the user is in a sleep state or an awake state, such as with motion.

Standard polysomnography (PSG) data collection, including electroencephalography, electromyography, and electrooculography, was performed using Compumedics Grael 4K PSG: electroencephalography (EEG) (Abbotsford, Virtoria, Australia), and sleep was scored according to the American Academy of Sleep (AASM) guidelines.

Physiological HFs were recorded and scored (2 μS/30 s rises in SC) by experienced scorers, according to gold standard methods: sternal SC (64 Hz) was collected via two 1.5 cm-diameter Ag/AgCl electrodes filled with 0.05 M potassium chloride Velvqachol/glycol gel placed on either side of the sternum (e.g., about 4 cm apart; a 0.5-V constant voltage circuit was maintained between them) using BioDerm SC Meter (model 2701; UFI, Morro Bay, Calif.).

Signals from a customized array of consumer-grade commercially available sensors (e.g., PPG: S/F SEN-11574-512 Hz; SC sensor: Grove 101020052-64 Hz; 3-axis motion sensor: NXP-FXOS8700-1024 Hz; and T sensor: TI-TMP36GT9Z-16 Hz) were collected from each women's wrist (M and PPG sensors on dorsal wrist, SC and T sensors on anterior wrist) and integrated with Compumedics recording system, using a multi-channel output card (40-Ch Digital-to-Analog Converter: A/D-AD5370).

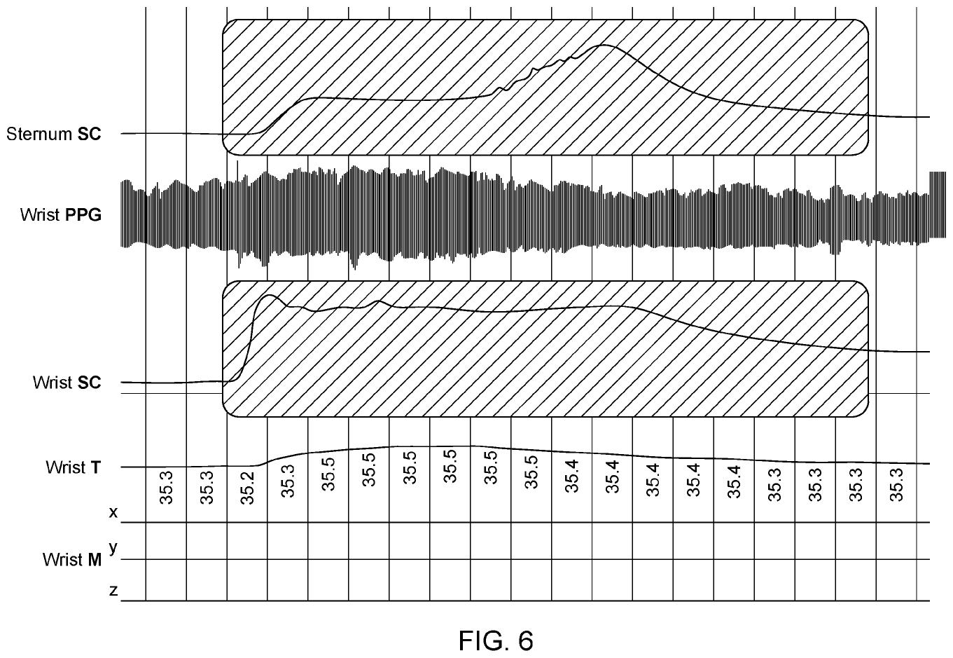

FIG. 6 illustrates an example graph of sensor signals from a plurality of sensor circuits of a system, in accordance with various embodiments. FIG. 6 shows the sensor signals from the gold standard sternum SC, and commercial grade available sensor signals from the wrist include PPG, SC, T, and N. The dashed lines illustrate a HF event, around eight minutes, for a participant female and the relevant sensor signals aligned.

The data collection was started around three hours before bedtime and continued overnight, until the morning awakening. Women slept in sound-attenuated and temperature-controlled bedrooms.

Features were extracted from the sensor signal data from the four wrist sensors (SC, PPG, T, M) and computed every fifteen seconds using a windowing approach. The left and right time windows were used for each feature computation. Distinct feature sets were obtained for SC, T, PPG, and M.

The following describes the processed feature sets from the multi-sensor circuit data. For the SC feature set, the HF onset output of a previously developed predictive data model was used as a feature. In some embodiments, the HF onset was designated as the ±two minutes around the HF predicted onset. In some embodiments, the previously developed predictive data model was implemented as described in M. Forouzanfar et al. “Automatic Detection of Hot Flash Occurrence and Timing from Skin Conductance Activity”, which is hereby incorporated herein in its entirety for its teaching. An SC+ feature set was developed that includes the SC feature set and the differential AUC of the SC sensor signal. To compute the differential, the AUC difference between the last 250 seconds and the current time window (±30 seconds) was taken. In addition, the derivative from step 8 of the previously developed predictive data model was doubled. The SC+ feature set aims to represent both slow and fast rising SC responses using the AUC and the SC feature set, respectively. For the T feature set, the temperature average differential of each female participant was computed between the prior and following 500 seconds. The feature aims to capture temperature changes before and after a HF event. For the PPG feature set, a FFT-based HR estimate was used. The HR estimate was averaged in two regions: 120 seconds before and after the time window. The differential of the HR change was used as a feature. The PPG feature set aims to capture HR changes before and after a HF event. For the M feature set, movements of the subject in the x, y, and z dimensions were captured. Each dimension was processed separately and the absolute maximum (AMD) (window ±30 seconds) was extracted for each dimension. For the x dimension, the raw AMD was used. For the y and z dimensions, the AMD differential between y, z, and x was used as features.

The features were then time-aligned with the HF expert annotations for prediction and evaluation (±90 second matching window). As all features were processed independently, the system enables sensor specific feature selection. The selected features were fed into a decision tree classifier, which makes a decision every fifteen seconds, whether or not the current time frame is a HF event by using the sensor signals from multiple sensor circuits. Using the decision output, HF regions were extracted for each participant.

The data was analyzed to evaluate: 1) SC features verses multi-sensor features in the HF classification performance; 2) HF classification performances as a function of whether the HF onset occurred during an awake state or a sleep state; and 3) the HF classification robustness in simulated noise environment.

First, the HF classification accuracy for the SC+ feature set verses the SC feature set was compared. The SC+ set was then augmented with the T, PPG, and M feature sets. The analysis of (1) was repeated with the same set of features (and same system) but for (2) the sleep and awake regions were compared as scored from PSG. The impact of the sensors on the two conditions were compared. The experiments of (1) were repeated with the same set of features and system, but for (3) corrupted signal with sensor contact loss was simulated. To similar sensor-contact loss (partial contact), fifteen percent (%) of the female participant's session region was randomly selected and assigned the lowest value of the signal, for each sensor signal independently.

In each of the analyses, the data was randomly split into two sets, with 80% of data for training the decision tree and 20% for evaluating the system. The process was repeated five times, cross-validation setup until all data was used for testing. For the decision tree training, a maximum depth of 6 was used (6 decisions from root to leaf) and with 5 minimum samples per leaf (a decision applies to 5 or more samples in the data; if the decision applies to less than 5 samples, the decision was discarded). To ensure similar specificity (96.5±1%) across analysis, the HF class was oversampled to 1:2 ratio between the HF regions and non-HF regions. HF performance was evaluated in terms of system sensitivity (percent of true positive) and specificity (percent of true negatives) in HF detection compared to the gold standard sternum SC expert evaluation.

The impact (e.g., contribution) of each sensor circuit was computing using Shapley values method, assigning the optimal impact to each sensor circuit given the consistency and additivity assumptions. For equal class representation, the analysis was run by a 1:1 ratio between the two classes.

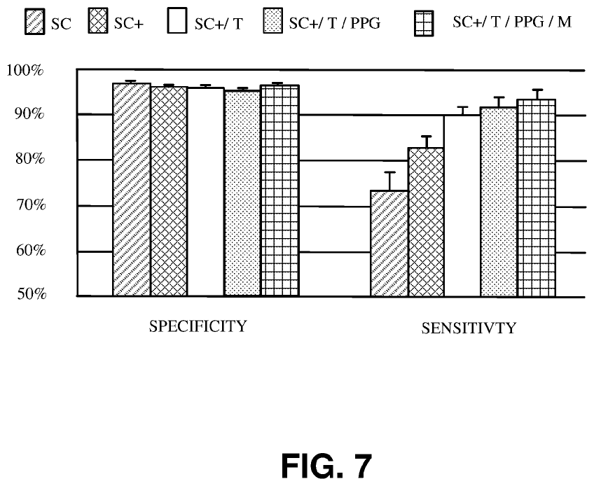

FIG. 7 illustrates an example graph of SC features as compared to multi-sensor features, in accordance with various embodiments. The left side of the graph shows the specificity and the right side shows the sensitivity of SC feature set, SC+ feature set, SC+ feature set with T feature set, SC+ feature set with T feature set and PPG feature set, and the SC+ feature set with T feature set, PPG feature set, and M feature set. The vertical bars represent mean and standard deviation. At 96.5% specificity, the SC+ feature set showed better HF sensitivity than the SC feature set (and +10.7% in sensitivity). The addition of T, PPG, and/or M feature sets to the SC+ feature set resulted in further improvements.

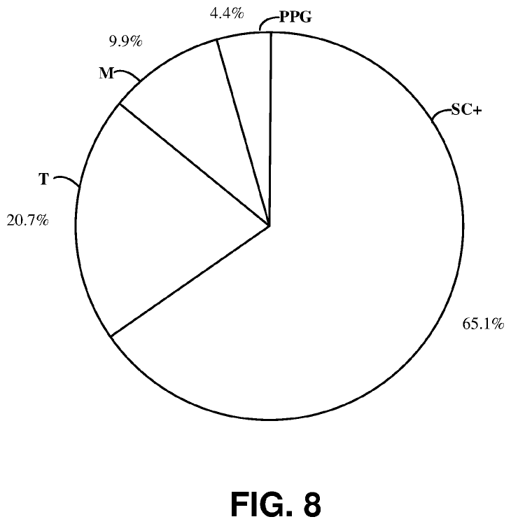

FIG. 8 illustrates an example graph of sensor circuit contributions, in accordance with various embodiments. The impact or contribution of each sensor circuit of the multi-sensor circuit system is shown in Shapley values of FIG. 8. While the SC signal accounts for the most variance in HF classification (˜65%), using the additional non-SC features further enhanced the classification performance.

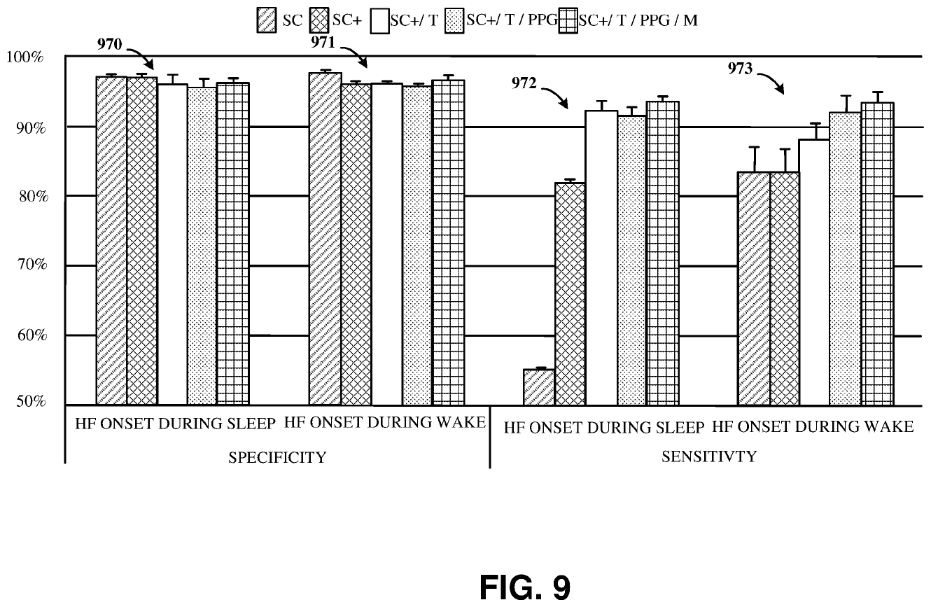

FIG. 9 illustrates an example graph of system performance for HF onsets during sleep and awake states, in accordance with various embodiments. The left side of the graph shows the specificity, at 970 and 971, and the right side shows the sensitivity of SC feature set, SC+ feature set, SC+ feature set with T feature set, SC+ feature set with T feature set and PPG feature set, and the SC+ feature set with T feature set, PPG feature set, and M feature set, at 972 and 973. The vertical bars represent mean and standard deviation. A greater contribution was observed for non-SC feature in the HF classification performance for HF with onsets occurring during sleep versus awake.

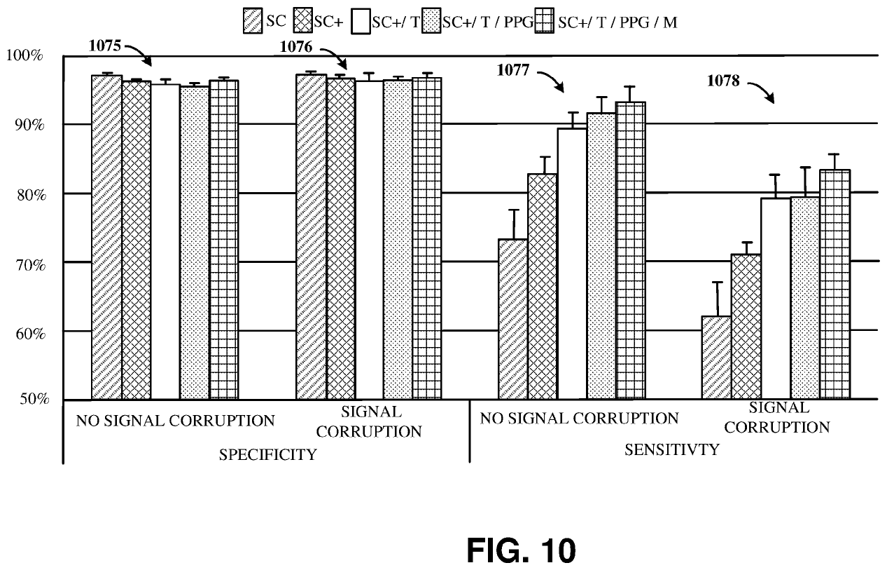

FIG. 10 illustrates an example graph of HF classification performance as a function of feature set and corrupted signals, in accordance with various embodiments. The left side of the graph shows the specificity, at 1075 and 1076, and the right side shows the sensitivity of SC feature set, SC+ feature set, SC+ feature set with T feature set, SC+ feature set with T feature set and PPG feature set, and the SC+ feature set with T feature set, PPG feature set, and M feature set and in conditions of reliable signals and corrupted signals, at 1077 and 1078. The vertical bars represent mean and standard deviation. When the signal were corrupted, the HF sensitivity performance deteriorated (below 75% when using SC features one) and the multi-sensor circuit approach at least partially compensated for the performance loss.

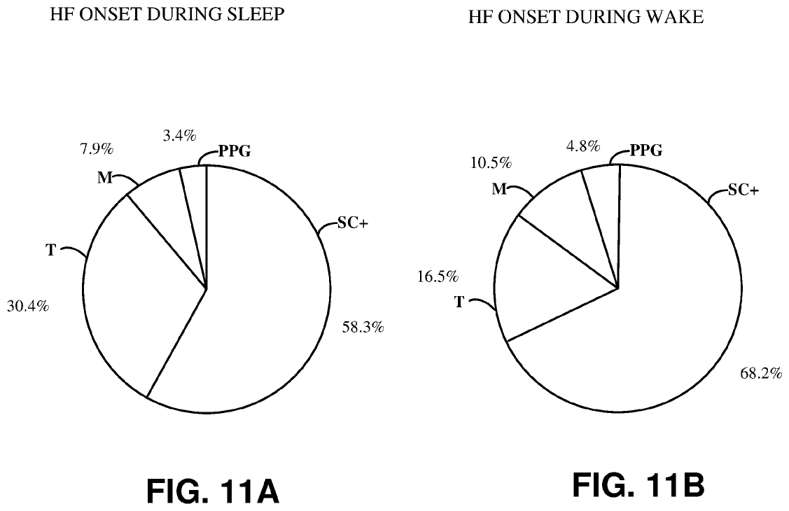

FIGS. 11A-11B illustrate example graph of sensor circuit contributions during awake and sleep states, in accordance with various embodiments in accordance with various embodiments. FIG. 11A illustrates the impact or contribution of each sensor circuit of the multi-sensor circuit system as shown in Shapley values during sleep states. FIG. 11B illustrates the impact or contribution of each sensor circuit of the multi-sensor circuit system as shown in Shapley values during awake states. From the Shapley values, the feature contributions in the HF classification was greater for the PPG, SC, and M feature sets, while it was less for the T feature set when comparing HFs with onset occurring during awake verses sleep states.

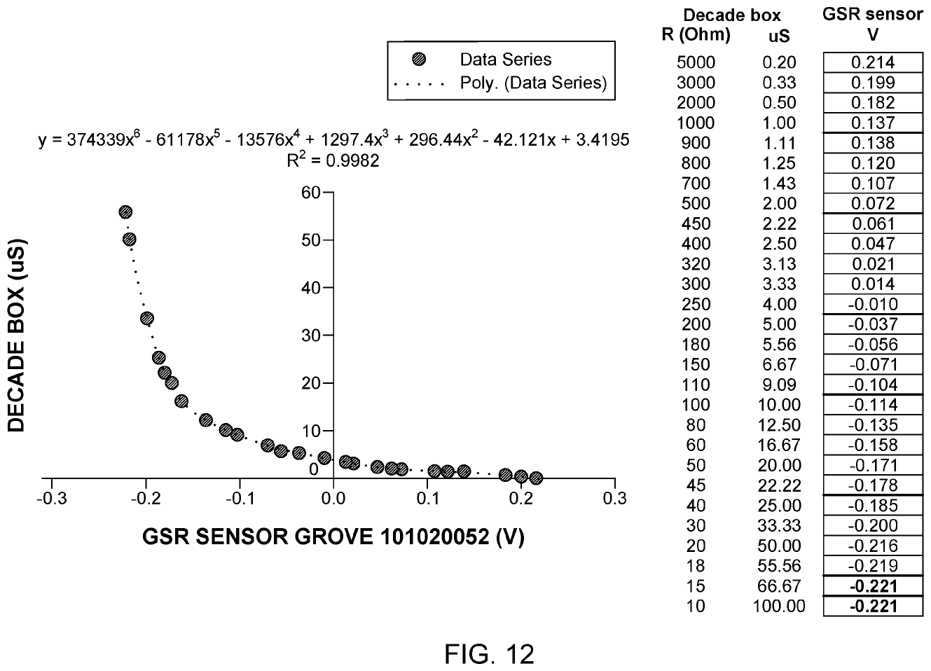

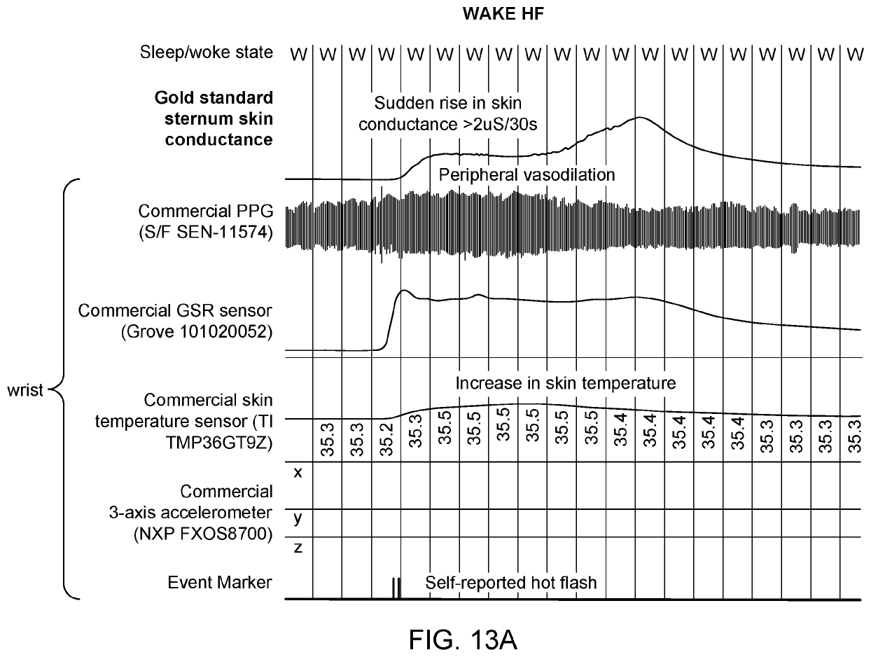

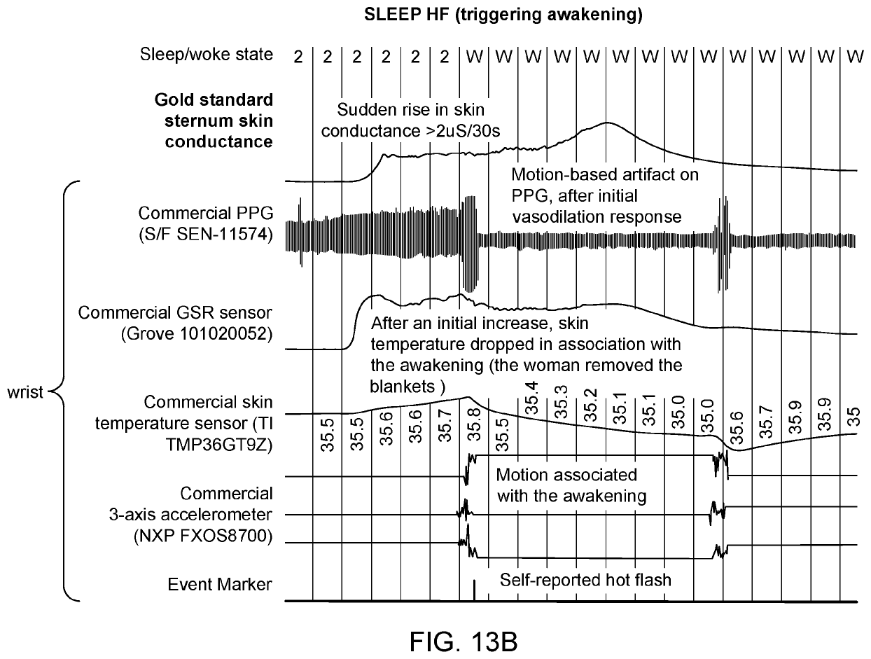

FIG. 12 illustrates an example graph of a commercial (GSR) sensor calibration and conversion, in accordance with various embodiments. As further illustrated by FIGS. 13A-13B, physiological HF were recorded via UFI Model 2701 BioDerm™ SC meters, showing fluctuation sternum SC (gold standard method) of >2 uS/30 s in a women undergoing laboratory testing. The HF morphology slightly varies across women. The SC peak usually occurs within a couple of minutes from its baseline and could be of different magnitude. Right after, the signal takes several minutes to return to the baseline levels. In some HFs, two consecutive peaks have been documented (15 to 20% of the cases, as reported by Bahr et al., before a baseline return).

The predictive data model used in accordance with embodiments of the present disclosure classifies HF events in real-time using consumer grade sensors, which can be implemented one or more multi-sensor wearable devices. In addition to SC fluctuations, a multitude of other physiological and behavioral changes (e.g., finger vasodilation, increases in HR, skin temperature) are integrated parts of the HF manifestation, and can contribute to both detection and full characterization (e.g., severity) of HFs. For example, early laboratory studies on samples of women explored the use of finger temperature in combination with HR or the combination of changes in finger temperature, blood volume, and changes in SC from different body locations in HF classification.

In a number of experimental embodiments, HFs physiology, and specifically the cardiovascular and autonomic changes associated with HFs onset, from >500 HFs recorded in different physiological states (e.g., wake and sleep) were investigated.

In a number of experimental embodiments, HR changes were compared to HF onsets. At the onset of the HF, HR increases and its variability reduces, while cardiac sympathetic activity and blood pressure drop. These changes follow distinct patterns, depending on whether or not the HF occurs during wake or sleep, and for the latter, whether HFs are associated or not with an arousal from sleep. More particularly, HR changes preceding HF onsets were identified in 80% of the cases from different data sources.

Initial feasibility testing was performed to evaluate system settings and conditions for accurate HFs measurement via consumer-grade sensors. In that effort, a customized data acquisition module was developed and integrated different signals (see FIGS. 13A-13B) from commercial sensors (PPG sensor: S/F SEN-11574, optical reflectance: Avago APDS-9008; GSR sensor: Grove 101020052; 3-axis motion sensor: NXP FXOS8700; Temperature sensor: TI TMP36GT9Z) with lab-grade Compumedics 4K High Definition dual platform PSG/EEG recording system (Abbotsford, Victoria, Australia), via multi-channel output card (40-Ch Digital-to-Analog Converter: A/D AD5370), to assure synchronization and facilitate multi-sensor signals comparison and integration. In order to capture the full range of SC fluctuations associated with HFs (0 to >50 uS), the sensitivity of the commercial GSR sensor was adjusted and sensor calibration was performed.

Different electrodes for GSR acquisition were tested, including Meditrace Ag/AgCl wet electrodes, matching the sensors used for gold standard SC monitoring (UFI Model 2701 BioDerm™ Skin Conductance Meters), and dry silver coated electrodes (similar to those used by Empatica wristbands (Empatica Inc., Boston, Mass.), a research-grade wearable originally targeting epilepsy via SC monitoring), as shown by FIG. 12.

The in-lab testing was on four midlife women, while awake in the evening and during sleep, recording a total of 47 HFs (based on expert evaluation of sternum SC morphology) with a combination of lab-grade and commercial sensors, from different wearable-target locations (e.g., wrist, neck, finger) using the integrated system.

FIGS. 13A-13B illustrate example graphs of a multi-sensor circuit approach verses a gold standard sternum SC approach, in accordance with various embodiments. The main outcome of the experiment was the scientifically-informed development of a multi-sensor HF identification (e.g., detection) based on feature extraction and data integration from commercial-grade sensors placed on the wrist (wearable-target location), by acknowledging different contextual information on which HFs occur and behavioral correlates. Particular attention was paid to the SC signal. Overall, similar behavior was observed between the commercial GSR (Grove 101020052) sensor SC output (wrist) and the research-grade UFI Model 2701 BioDerm™ Skin Conductance Meters (sternum) (see FIGS. 13A-13B, for an example of a multi-sensor HF recording), as well as expected HF correlates from wrist commercial sensors (e.g., peripheral vasodilation from PPG, and derived HR increases in association with the HF onset). The experiments also confirmed the use of multi-sensor circuit integration in HF characterization to account for different psychophysiological stated in which HF occurs (wake/sleep) and behavioral correlates (patient behavior associated to HF occurrence/perception). For example, if physiological rises in skin temperature are expected in association with the onset of a HF, then a drop in skin temperature following a HF during sleep can reflect a combination of heat loss due to increased vasodilation and removal of the blankets after waking up from a sleep HF, with a larger drop expected in the latter case as a different behavior in the case of HFs occurring during sleep (if the participant wakes up), reflecting a combination of HF-related physiological response and behavior (in FIG. 13B, when the patient removed the blankets after waking up from a sleep HF results in a drop in skin temperature).

More specifically, FIGS. 13A-13B illustrate example graphs of HF detecting using a gold standard sternum SC and additional consumer grade sensors, in accordance with various embodiments. This gold standard was compared to integrating features from multiple commercially-available sensors placed on the wrist. In particular, four target sensors were combined: (1) SC, (2) T, (3) IMU, and, (4) PPG. A variety of functions and contextualization time windows per feature per sensor were used. The feature diversity provides robustness, especially when noise impacts sensor reliability.

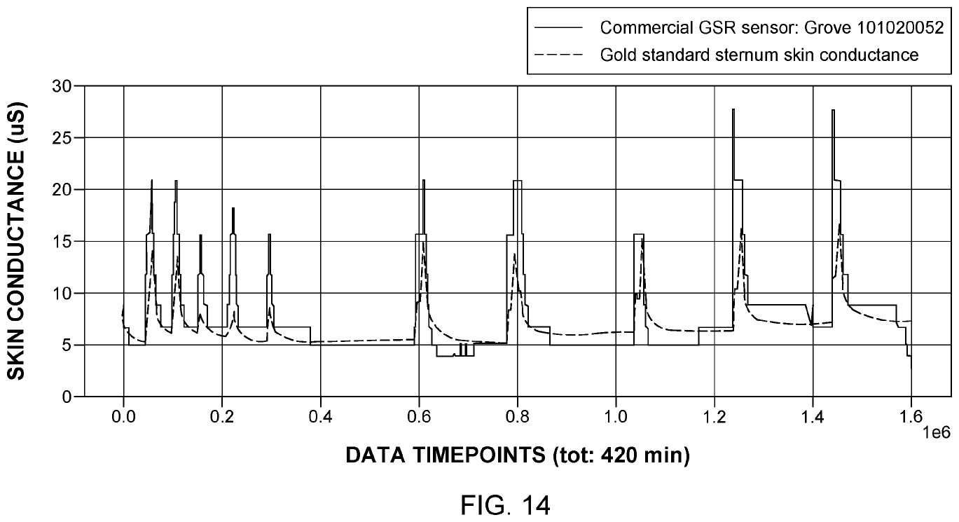

FIG. 14 illustrates an example graph of a commercial GSR sensor (applied on the wrist) compared to a gold standard sternum SC approach, in accordance with various embodiments.

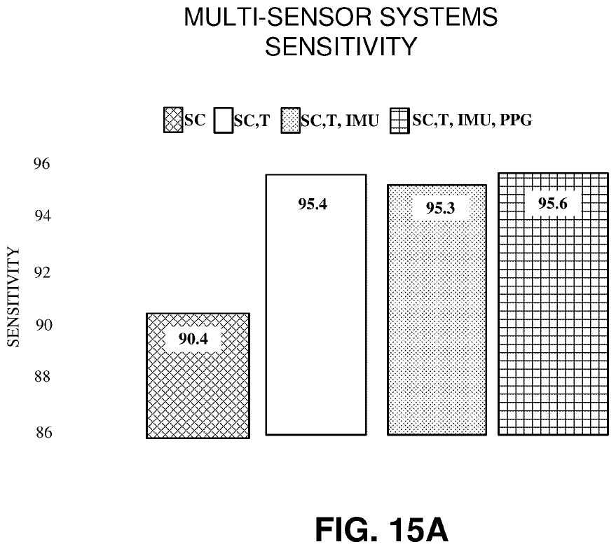

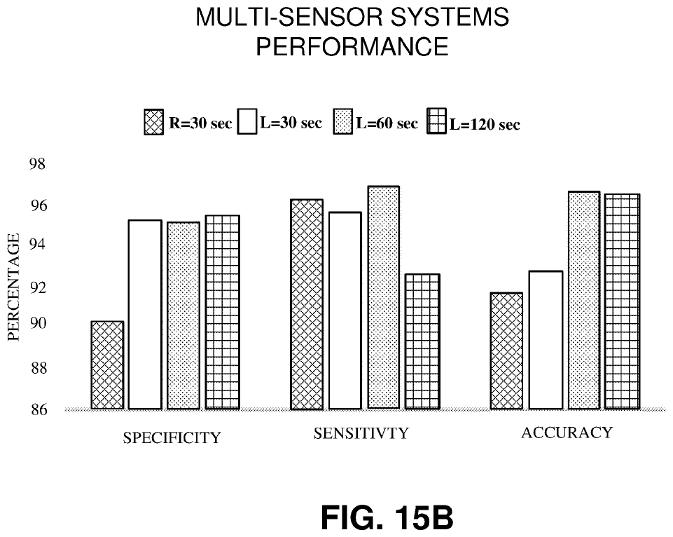

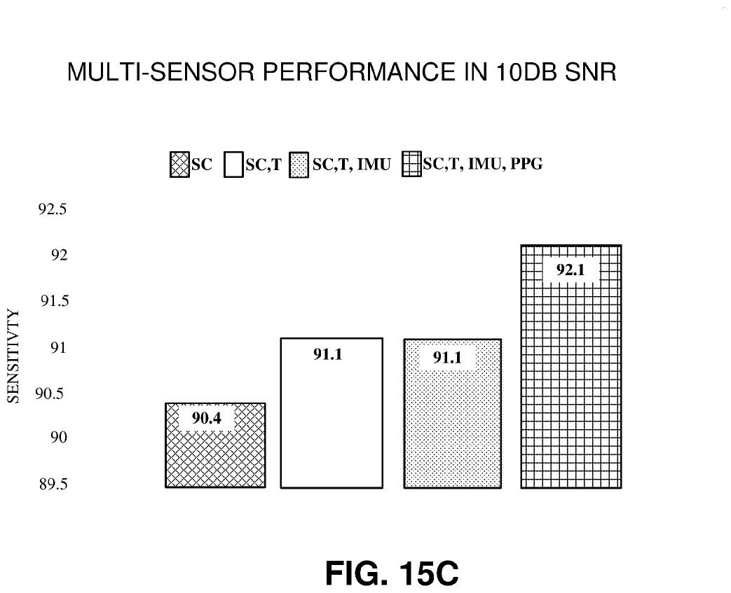

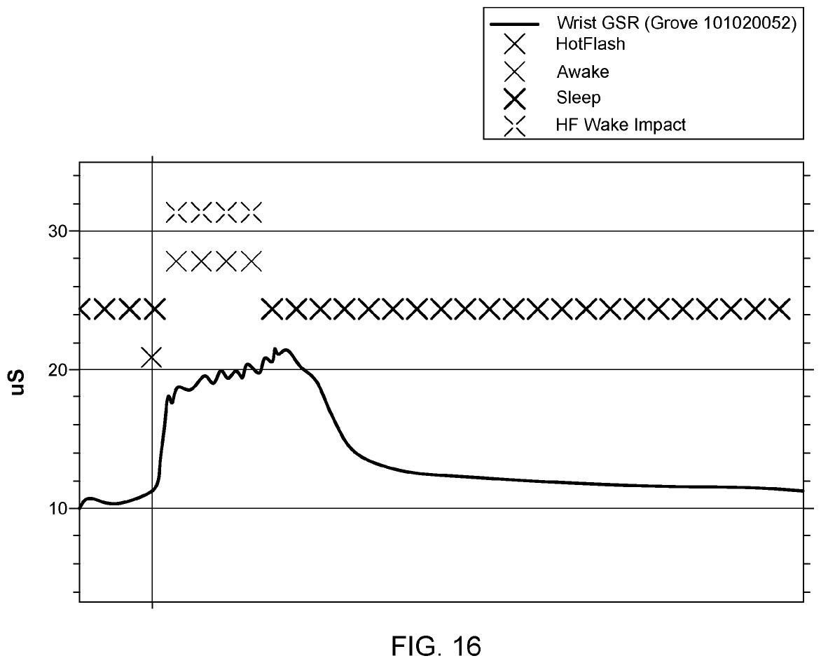

FIGS. 15A-15C illustrate example graphs showing experimental results, in accordance with various embodiments. Experiments were conducted on three participants for HF detection. In this setup, HFs were detected using four sensor combinations: (1) SC, (2) SC+T, (3) SC+T+IMU, and (4) SC+T+IMU+PPG. The sensitivity and specificity were calculated every thirty seconds. FIG. 16A shows the sensitivity (%) when specificity is higher than 90%. As shown, (1) the SC sensors account for the majority of the predictor's sensitivity and (2) additional sensors increase the sensitivity above 95%. Thus, multi-sensor circuit systems capture information unavailable to SC-based systems.

The multi-sensor circuit contextual features impact real-time and low-resource processing critical in commercial systems. The impact of contextual information processing on performance was assessed. FIG. 15B shows the performance trade-offs for the SC+T+IMU+PPG system. Note that the amount of left and right by L=XX seconds (e.g., 30, 60, and 120 seconds) and R=XX seconds (e.g., 30 seconds) respectively. It was observed that ninety seconds of context impacts accuracy by more than 5%. On the other hand, longer context delays the real-time output and increases resource and battery requirements. Thus, satisfying commercial-systems constraints relies on selecting and optimizing the context.

As opposed to lab-grade systems, real-world commercial systems operate under diverse environmental, sensor and behavior conditions. One common condition is noisy or disconnected sensors. Using preliminary data, the importance of multi-sensor circuit systems when sensors are noisy was analyzed. Noisy conditions impact were simulated by adding white noise such that there is 18 dB signal-to-noise ratio. FIG. 15C shows that using multiple sensors improves sensitivity by 2% for specificity greater than 95%. Thus, even though noise was added to all four sensor circuits, the multi-sensor circuit system increases robustness when compared to the SC system.

The experimental data indicates that the multiple sensors provide system robustness and trade-offs between (1) real-time speed, (2) processing resources, and, (3) sensitivity performance. The array of sensors used in HFs detection is also currently used by wearable devices to track biology of the users (e.g., autonomic function, menstrual cycle), behaviors (e.g., sleep, physical activity), and environment. Thus, a multi-sensor circuit approach to HF characterization and management has the potential of integrating HF measurement with other relevant menopause and HFs-related aspects, such as measuring the impact of HFs on other bio-systems (e.g., sleep), and investigating potential HF-triggers (e.g., changing in environmental temperature).

For example, different patterns of HFs occur at night, differently impacting user's sleep. Despite not all the objectively-recorded HFs being associated with sleep disturbances, the data shows that about 70% of HFs wake the user up and are responsible for about 30% of total wake time at night. The predictive data model in accordance with various embodiments can be used to simultaneously measures sleep/wake state and HF occurrence, calculating the amount of wake associated with the HF occurrence, the implementation of the HF-impact index (reflecting the direct impact of HFs on sleep).

FIG. 16 illustrates an example graph of performance of the predictive data model, in accordance with various embodiments.

Various embodiments are implemented in accordance with the underlying Provisional Application (Ser. No. 63/062,692), entitled “Multi-Sensor System and Method for Hot Flash Detection and Mitigation,” filed Aug. 7, 2020, to which benefit is claimed and which are both fully incorporated herein by reference for their general and specific teachings. For instance, embodiments herein and/or in the provisional application can be combined in varying degrees (including wholly). Reference can also be made to the experimental teachings and underlying references provided in the underlying provisional application. Embodiments discussed in the Provisional Application are not intended, in any way, to be limiting to the overall technical disclosure, or to any part of the claimed disclosure unless specifically noted.

Although described or shown with respect to one embodiment, the features and elements so described or shown can apply to other embodiments. It will also be appreciated by those of skill in the art that references to a structure or feature that is disposed “adjacent” another feature may have portions that overlap or underlie the adjacent feature.

Terminology used herein is for the purpose of describing particular embodiments only and is not intended to be limiting of the invention. For example, as used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as“/”.

Although various embodiments are described above, any of a number of changes may be made to various embodiments without departing from the scope of the invention as described by the claims. For example, the order in which various described method steps are performed may often be changed in alternative embodiments, and in other alternative embodiments one or more method steps may be skipped altogether. Optional features of various device and system embodiments may be included in some embodiments and not in others. Therefore, the foregoing description is provided primarily for exemplary purposes and should not be interpreted to limit the scope of the invention as it is set forth in the claims.

The skilled artisan would recognize that various terminology as used in the Specification (including claims) connote a plain meaning in the art unless otherwise indicated. As examples, the Specification describes and/or illustrates aspects useful for implementing the claimed disclosure by way of various circuits or circuitry which may be illustrated as or using terms such as blocks, modules, device, system, unit, controller, and/or other circuit-type depictions. Such circuits or circuitry are used together with other elements to exemplify how certain embodiments may be carried out in the form or structures, steps, functions, operations, activities, etc. For example, in certain of the above-discussed embodiments, one or more modules are discrete logic circuits or programmable logic circuits configured and arranged for implementing these operations/activities, as may be carried out in the approaches shown herein. In certain embodiments, such a programmable circuit is one or more computer circuits, including memory circuitry for storing and accessing a program to be executed as a set (or sets) of instructions (and/or to be used as configuration data to define how the programmable circuit is to perform), and process is used by the programmable circuit to perform the related steps, functions, operations, activities, etc. Depending on the application, the instructions (and/or configuration data) can be configured for implementation in logic circuitry, with the instructions (whether characterized in the form of object code, firmware or software) stored in and accessible from a memory (circuit).

Various embodiments described above, may be implemented together and/or in other manners. One or more of the items depicted in the present disclosure can also be implemented separately or in a more integrated manner, or removed and/or rendered as inoperable in certain cases, as is useful in accordance with particular applications. In view of the description herein, those skilled in the art will recognize that many changes may be made thereto without departing from the spirit and scope of the present disclosure.

What is claimed is:

1. A system comprising:
a plurality of sensor circuits configured to obtain a plurality of sensor signals associated with a user; and
processor circuitry in communication with the plurality of sensor circuits and configured to:
extract features from the plurality of sensor signals obtained by the plurality of sensor circuits;
align the extracted features to a common time point;
identify a hot flash (HF) event for the user using a predictive data model indicative of a probability of the HF event occurring for the user at a date and time and based on the aligned extracted features; and
communicate a message indicative of the HF event to the user.

2. The system of claim 1, wherein the plurality of sensor circuits include two or more sensor circuits selected from a photoplethysmogram (PPG) sensor, a skin conductance (SC) sensor, a temperature (T) sensor, and a motion (M) sensor.

3. The system of claim 1, wherein the processor circuitry is configured to characterize a level or presence of the HF event based on the extracted features.

4. The system of claim 1, wherein the message includes at least one of the identification of the HF event and an intervention action for the HF event, and the processor circuitry is configured to identify the HF event in real-time.

5. The system of claim 1, wherein the processor circuitry is configured to:
identify a psychophysiological state of the user based on the extracted features; and
identify, using the predictive data model, a pattern of physiological measurements indicative of the probability of the HF event occurring based on the aligned extracted features and the psychophysiological state of the user.

6. The system of claim 5, wherein the psychophysiological state includes a sleep state or an awake state, and the processor circuitry is configured to calculate an amount of awake time associated with the HF event.

7. The system of claim 1, wherein the processor circuitry is configured to:
align the extracted features to the common time point based on a plurality of different time windows associated with the plurality of sensor circuits; and
weigh each of the extracted features based on an impact of the extracted features on the probability of the HF event occurring.

8. The system of claim 1, wherein the predictive data model includes a plurality of sub-models, and each of the plurality of sub-models are associated with a respective sensor circuit of the plurality and provide an output score indicative of the probability of the HF event occurring for the user based on the extracted features from the respective sensor signal obtained by the respective sensor circuit; and
the processor circuitry is configured to combine the output scores from the plurality of sub-models to identify the HF event.

9. The system of claim 1, wherein the processor circuitry is configured to combine the extracted features from the plurality of sensor signals into a vector and input the vector to the predictive data model to produce an output score indicative of the probability.

10. The system of claim 9, wherein the processor circuitry is configured to generate a decision tree structure to combine the extracted features, to produce the output score based on the combined extracted features, and to:
identify whether the HF event is occurring or not at a plurality of time points;
detect consecutive identified HF events; and
convert the consecutive identified HF events into a HF region.

11. The system of claim 1, wherein the processor circuitry is configured to receive feedback data in response to the communicated message, the feedback data being indicative of at least one of a user confirmation of the HF event, a user denial of the HF event, and a severity of the HF event.

12. A non-transitory computer-readable storage medium comprising instructions that when executed cause processor circuitry to:
extract features from a plurality of sensor signals associated with a user, the plurality of sensor signals being obtained by a plurality of sensor circuits;
identify a HF event for the user using a predictive data model indicative of a probability of the HF event occurring for the user at a date and time based on the extracted features; and
revise the predictive data model based on feedback data indicative of an impact of the HF event on the user.

13. The non-transitory computer-readable storage medium of claim 12, further including instructions executable to align the extracted features to a common time point based on a plurality of different time windows of the plurality of sensor circuits used to obtain the plurality of sensor signals.

14. The non-transitory computer-readable storage medium of claim 12, further including instructions executable to receive the feedback data, the feedback data including at least one of:
a user confirmation of the HF event, a user denial of the HF event, a severity of the HF event, and an impact of an intervention action on the HF event.

15. The non-transitory computer-readable storage medium of claim 14, wherein each feature of the extracted features is associated with a weight indicative of an impact of the respective feature on the probability of the HF event, and wherein the instructions to revise the predictive data model include instructions executable to perform at least one of:
adjust a first weight associated with a first feature of the extracted features;
adjust the first weight and a second weight associated with the first feature for different psychophysiological states of the user; and
adjust an intervention action for additional HF events.

16. The non-transitory computer-readable storage medium of claim 12, further including instructions executable to communicate a message indicative of the HF event to the user, wherein the message indicates least one of an occurrence of the HF event, a prediction of the occurrence of the HF event, and an intervention action for the HF event.

17. A system comprising:
a plurality of sensor circuits configured to obtain a plurality of sensor signals associated with a user over a plurality of different time windows; and
processor circuitry in communication with the plurality of sensor circuits and configured to:
extract features from the plurality of sensor signals;
align the extracted features to a common time point based on the plurality of different time windows associated with the plurality of sensor circuits;
track a psychophysiological state of the user based on the aligned extracted features;
track a plurality of HF events for the user using a predictive data model indicative of probability of a HF occurring for the user at a date and time based on the aligned extracted features and the tracked psychophysiological state of the user; and
communicate a message indicative of the plurality of HF events to the user.

18. The system of claim 17, wherein the tracked psychophysiological state is associated with a sleep state or an awake state of the user, and the processor circuitry is configured to calculate an amount of awake time associated with at least one of the plurality of HF events based on the tracked psychophysiological state.

19. The system of claim 17, wherein the processor circuitry is configured to revise the predictive data model based on feedback data indicative of an impact of the plurality of HF events on the user, the revision including adjusted weights for the extracted features as associated with the tracked psychophysiological state.

20. The system of claim 17, wherein the predictive data model includes weights for each of the extracted features and for different psychophysiological states, each weight being associated with the probability of the HF occurring at the date and time.
